(19) United States
(12) Patent Application Publication
(10) Pub. No.: US 2025/0010026 A1
(43) Pub. Date: Jan. 9, 2025

(54) CATHETER ASSEMBLY

(71) Applicant: ConvaTec Limited, Flintshire (GB)

(72) Inventors: Oliver Pfleger, Spital (GB); Lukasz Natkaniec, Munchen (DE); Phillip Emrich, Munchen (DE)

(21) Appl. No.: 18/891,659

(22) Filed: Sep. 20, 2024

Related U.S. Application Data
(63) Continuation of application No. PCT/GB2023/050694, filed on Mar. 20, 2023.
(60) Provisional application No. 63/394,702, filed on Aug. 3, 2022.

(30) Foreign Application Priority Data
Mar. 21, 2022 (GB) 2203882.2
Aug. 12, 2022 (GB) 2211844.2

(51) Int. Cl. A61M 25/00; A61M 39/22

(52) U.S. Cl. CPC A61M 25/0017; A61M 25/0009; A61M 25/002; A61M 39/223; A61M 2205/3331; A61M 2205/584; A61M 2207/00; A61M 2209/06; A61M 2210/1096

(57) ABSTRACT

A catheter assembly that has a catheter having a proximal end for insertion into the body and a distal end, a valve assembly, and a fluid collection bag arranged to receive fluid from the distal end of the catheter via the valve assembly. The valve assembly has a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag. The catheter may be a male urinary catheter. Colour may be used to indicate the configuration of the valve assembly.

### CROSS-REFERENCE TO RELATED DISCLOSURE

The present disclosure is a Continuation of International Application No. PCT/GB2023/050694 filed Mar. 20, 2023 and claims the benefit of GB2203882.2 filed Mar. 21, 2022; GB2211844.2 filed Aug. 12, 2022; and U.S. 63/394,702 filed Aug. 3, 2022, the disclosures of which are incorporated herein by reference in entirety.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

To reduce the risk of damage to the urinary tract and improve comfort while using the catheter, it is also important they are activated before use for example through wetting of the catheter by a wetting fluid. However, once wetted and/or used a catheter can be slippery and difficult to handle. This can make handling and re-packaging of the catheter after use difficult and unhygienic, as the catheter may inadvertently contact the user's hands, clothing or personal items. Existing intermittent catheters disclose continuous handling sleeves that cover the entire length of the catheter. While these provide some assistance to handling, the sleeve can make the catheter more difficult to wet as wetting fluid may not be able to easily reach all parts of the catheter.

To allow self-catheterisation as required, closed catheter assemblies collect and retain urine using a fluid collection bag, which removes the need for urine to pass directly into a toilet. To provide a fluid collection bag that is discreet prior to use but also able to store a suitable volume of liquid, fluid collection bags can be substantially 2D prior to use, consisting of two panels joined at their periphery. However, this can cause problems for the user as they must support or hold the fluid collection bag to ensure that it fills during use.

Once used, a fluid collection bag is heavy and bulky as it is filled with liquid. To allow the bag to be carried and disposed of more discretely and easily, existing bags may be provided with a drain to allow them to be emptied into a toilet. However, existing drains can either leak or are difficult to open which potentially results in inadvertent or uncontrolled release of liquid from the bag. In addition, the drain can add to the bulk of the catheter assembly which makes it less discreet and consequently less desirable for the user.

To provide discrete and portable catheter assemblies, packaging may be provided to contain the catheter and any other equipment such as wetting reservoirs or sachets and fluid collection bags before and/or after use. However, where liquid is contained within a sachet, reservoir or bag within the packaging, any release (intentional or otherwise) of this may cause liquid to accidentally leak out onto parts of the catheter assembly that the user has to touch-such as a handling element, or onto the packaging causing problems for the user.

It is an object of embodiments of the present invention to at least partially overcome or alleviate the above problems and/or to provide an improved packaged catheter assembly.

### SUMMARY OF THE INVENTION

In broad terms, the present invention concerns a catheter assembly comprising a catheter. The catheter assembly may be a urinary catheter assembly. In addition, the present invention also concerns a packaged catheter assembly comprising a pouch and the catheter assembly, wherein the pouch is configured to contain the catheter assembly within it. The pouch may be configured to contain within it any one or more of: a catheter; a fluid reservoir; a fluid collection bag; and a sleeve, any of which may be as defined in the statements of invention below.

In a broad aspect, there is provided a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter; and a second configuration in which the valve assembly permits fluid flow in a direction from the fluid collection bag towards the catheter. Preferably, in the first configuration the valve assembly permits fluid flow in a direction from the catheter towards the fluid collection bag. In the second configuration, the valve assembly may inhibit fluid flow in a direction from the catheter towards the fluid collection bag.

Accordingly, a first aspect of the present invention provides a urinary catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

Advantageously, the two configurations of the valve assembly allow the user to efficiently fill the fluid collection bag and store fluid within it, as well as drain the bag in a controlled manner without the need to tear or open the bag. In addition, the valve assembly removes the need for a separate inlet and outlet in the fluid collection bag as a single opening in the fluid collection bag can operate as both the inlet and outlet, this reduces the manufacturing cost and complexity of the fluid collection bag. In addition, it makes the bag simpler to use and more robust.

Accordingly in one embodiment there is provided a packaged catheter assembly comprising a pouch and the catheter assembly of the first aspect, wherein the pouch is configured to contain the catheter assembly within it.

Accordingly in one embodiment there is provided a packaged catheter assembly comprising a pouch and a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag wherein the pouch is configured to contain the catheter assembly within it.

The catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the intermittent catheter is formed of a polyolefin material, in particular polyethylene and/or polypropylene.

Preferably, the intermittent catheter is formed of a thermoplastic elastomeric material.

The catheter may be an intermittent catheter. The catheter may be a urinary catheter. The catheter may be a male urinary catheter. The catheter may be a female urinary catheter.

Accordingly in a preferred embodiment there is provided a catheter assembly comprising a male urinary catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

The valve assembly may be provided in the first configuration. For example, the catheter assembly may be provided in the first configuration in a sealed pouch. The valve assembly may be configured to move from the first configuration to the second configuration. The valve assembly may be configured to move from the second configuration to the first configuration. The valve assembly may be configured to move reversibly between the first and second configurations. Thus, the valve assembly is reversible between the first and second configurations which allows the user to effectively re-seal the fluid collection bag if necessary. This can help prevent further leaks after draining the bag which can be common in bags where an outlet is opened irreversibly (for example by tearing the bag) and fluid lining the inside of the fluid collection bag slowly collects and flows out.

The valve assembly may be configured to resist movement from the second configuration to the first configuration. The valve assembly may be configured to resist movement from the first configuration to the second configuration. The valve assembly may remain in the first or second configuration. Thus, the valve assembly may not be reversibly switched between the first and second configurations as once in a configuration it resists movement back to the other configuration. This can help reduce the risk of inadvertently changing the configuration multiple times when only one change of configuration is required.

The valve assembly may comprise two passageways: a proximal passageway and a distal passageway. The proximal passageway may be configured to provide a fluid communication between the valve assembly and the catheter. The distal passageway may be configured to provide a fluid connection between the valve assembly and the fluid collection bag. The valve assembly may comprise a valve housing. The proximal and distal passageways may connect to opposite sides of the valve housing. The valve housing may be any suitable shape such as cuboid, spherical, cylindrical or irregularly shaped, preferably it is cylindrical. The proximal and distal passageways may connect to a side wall of the valve housing. The valve housing may have a diameter (or width) that is at least 1 cm, 1.5 cm, 2 cm or 3 cm. The valve housing may have a diameter that is no more than 4 cm, 3 cm, 2 cm or 1.5 cm. Preferably, the valve housing has a diameter of 3 cm. The valve housing may have an aspect ratio (length:width/diameter) of at least 1:1, 2:1 or 3:1. The valve housing may have an aspect ratio (length:width/diameter) of no more than 3:1, 2:1 or 1:1. Preferably, the valve housing has an aspect ratio of (length:width/diameter) 2:1. Thus, the valve assembly can be conveniently be placed in-line with the catheter and fluid collection bag via the passageways.

Each passageway may extend away from the valve housing by a distance that is no more than twice the diameter/width of the valve housing, and preferably a distance that is no more than the diameter/width of the valve housing. Thus, the valve assembly itself remains compact and the passageways are less likely to be damaged by bending forces experienced during use/manipulation of the catheter and fluid collection bag.

Each passageway may be tubular. Each passageway may have an internal diameter that is the same size or greater than an internal diameter of the catheter. Thus, the passageways have a lower fluid resistance than the catheter and do not unnecessarily inhibit the speed of fluid flow between the catheter and fluid collection bag.

The valve assembly may comprise a user operable control. The user operable control may be configured to move the valve assembly from the first configuration to the second configuration. The user operable control may be configured to move the valve assembly from the second configuration to the first configuration. The user operable control may be configured to reversibly move the valve assembly between the first and second configurations. Alternatively, the user operably control may not be reversible and may irreversibly switch the valve assembly from the first to the second configuration. The user operable control may comprise any suitable user actuable input such as a switch, button, lever, slider, knob. Preferably, the user operable control comprises a button. The button may be any suitable shape or size but is preferably the same shape as one side or face of the valve housing. The button preferably covers a majority of a side or face of the valve housing. Where the valve housing is cylindrical, the button is preferably cylindrical. One end of the valve housing may be an open end. The button may fill the open end of the valve housing. The button may be configured to move axially with respect to the (cylindrical) valve housing. The button may have an extended configuration. The extended configuration of the button may correspond to the first configuration of the valve assembly. In the extended configuration the button may extend beyond the open end of the valve housing. The button may have a depressed configuration. The depressed configuration of the button may correspond to the second configuration of the valve assembly. In the depressed configuration the button may be closer to the open end of the valve housing than when it is in the extended configuration. Preferably in the depressed configuration the button is flush with the open end of the valve housing. The valve assembly is therefore easy to use as the user can simply press down anywhere on one side of the valve assembly to operate the valve assembly, this is particularly useful where a user has reduced manual dexterity.

The user operable control may be biased to move the valve assembly to the first configuration. The user operable control may comprise a biasing element, for example a spring or other material/object that can be resiliently deformed by a user's hand or grip strength. The biasing element may be configured to bias the user operable control to move the valve assembly to the first configuration. The user operable control may be biased to keep the valve assembly in the first configuration. When the valve assembly is in the first configuration, the biasing element may be configured to bias the user operable control to keep the valve assembly to the first configuration.

The user operable control may be biased to move the valve assembly to the second configuration. The biasing element may be configured to bias the user operable control to move the valve assembly to the second configuration. The user operable control may be biased to keep the valve assembly in the second configuration. When the valve assembly is in the second configuration, the biasing element may be configured to bias the user operable control to keep the valve assembly to the second configuration. Thus, the user operable control helps to ensure that inadvertent switching between the first and second configurations is avoided.

The user operable control may comprise a switching point. The biasing element may be configured to reverse the bias of the user operable control when the user operable control is moved to and/or past the switching point. When the valve assembly is in the first configuration, the biasing element may be configured to bias the user operable control to move the valve assembly to the second configuration when the user operable control is moved to and/or past the switching point. When the valve assembly is in the second configuration, the biasing element may be configured to bias the user operable control to move the valve assembly to the first configuration when the user operable control is moved to and/or past the switching point. The switching point may correspond to the depressed configuration of the button. The switching point may correspond to a position in which the button is depressed further than the depressed configuration. Thus, the switching point ensures the valve assembly is only switched between the first and second configurations when the user intends which reduces the risk of leaks through the valve assembly.

The valve assembly may be configured to provide feedback as it switches between the first and second configurations, for example an audible/tactile output such as a “click” and/or a change of visual appearance. An audible and/or tactile output may be provided by any suitable means such as via a protrusion on the inside of the valve housing that overrides a detent on the button as the button moves with respect to the valve housing.

The valve assembly may be configured to indicate if it is in the first or second configuration. The user operable control may be configured to change the appearance of at least part of the valve assembly as the valve assembly is moved between the first and second configurations. The button may comprise a side wall that is a different colour or shade of colour from the valve housing. The side wall of the button may be visible when the button is in the extended configuration/the valve assembly is in the first configuration. The side wall of the button may be hidden by the valve housing when the button is in the depressed configuration/the valve assembly is in the second configuration. Thus, button provides a visual reference to the state of the valve assembly.

Accordingly in one embodiment there is provided a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag, wherein the valve assembly is configured to move from the first configuration to the second configuration and/or from the second configuration to the first configuration, wherein the valve assembly comprises a user operable control configured to move the valve assembly from the first from the first configuration to the second configuration and/or from the second configuration to the first configuration, wherein the user operable control comprises a button, wherein the valve assembly comprises a valve housing and the button comprises a side wall that is a different colour or shade of colour from the valve housing.

The valve housing may comprise a window. The window may be located in a side wall of the valve housing. The window may be configured to change appearance depending on if the valve assembly is in the first configuration or second configuration. The window may be located equidistance from each of the passageways. The window may be elongate. The window may comprise a length in a direction parallel to the axis of the (cylindrical) valve housing. The window may have a length that is approximately half the diameter/width of the valve housing. The window may have an aspect ratio (length:width) or 1:1, 2:1, 3:1 or 4:1, preferably 2:1. The window may be any suitable shape, such as circular, semi-circular, triangular, rectangular, or irregularly shaped, preferably, the window is stadium-shaped, that is rectangular semicircles replacing two opposite sides. The button may comprise a side wall with two regions which are visually distinct from one another, for example they may be a different colour, a different shade of colour and/or comprise different markings on them. One of the regions may correspond to the position of the window when the button is in the extended configuration and the other region may correspond to the position of the window when the button is in the depressed configuration. One region may be the same colour as the side wall of the button and the other may be a different colour/shade of colour as the side wall of the button. Alternatively, both regions may be a different colour or shade of colour from the side wall of the button. Thus, a window can provide a simple visual reference to the state of the valve assembly as a specific region of the button is visible through the window depending on the state of the button and therefore the valve assembly.

Accordingly in one embodiment there is provided a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag wherein the valve assembly comprises a valve housing and the valve housing comprises a window, wherein the window may be configured to change appearance depending on if the valve assembly is in the first configuration or second configuration.

A top end of the button, that is the end that is outside the valve housing, may comprise an activation marker. The activation marker may comprise a visual indication to signal to the user how to use the button, for example it may comprise the word “PUSH” written on the button. The side wall of the button may also comprise an activation marker, for example an arrow pointing in the direction the button should be depressed. Thus, the operation of the button is intuitive for the use.

The valve assembly may comprise a valve body inside the valve housing. The valve body may be fluidly connected to the catheter (e.g. via the proximal passageway). The valve body may be fluidly connected to the fluid collection bag (e.g. via the distal passageway). The valve body may be connected inline between the catheter and fluid collection bag (e.g. via the proximal and distal passageways). The valve body may be configured to inhibit fluid flow in a direction from the fluid collection bag towards the catheter when the valve assembly is in the first configuration. The valve body may be configured to permit fluid flow in a direction from the catheter towards the fluid collection bag when the valve assembly is in the first configuration. The valve body may be configured to permit fluid flow in a direction from the fluid collection bag towards the catheter when the valve assembly is in the second configuration. The valve body may be configured to inhibit fluid flow in a direction from the catheter towards the fluid collection bag when the valve assembly is in the second configuration. The valve body may be coupled to the user operable control. Thus, the valve body enables the valve assembly to function as required.

The valve body may be configured to inhibit fluid flow in a direction from the distal passageway towards the proximal passageway when the valve assembly is in the first configuration. The valve body may be configured to permit fluid flow in a direction from the proximal passageway towards the distal passageway when the valve assembly is in the first configuration. The valve body may be configured to permit fluid flow in a direction from the distal passageway towards the proximal passageway when the valve assembly is in the second configuration. The valve body may be configured to inhibit fluid flow in a direction from the proximal passageway towards the distal passageway when the valve assembly is in the second configuration.

The valve body may be any suitable fluidic system to enable the valve assembly to operate as described above. The valve body may comprise a first check valve. The first check valve may be connected to the catheter (e.g. via the proximal passageway). The first check valve may be connected to the fluid collection bag (e.g. via the distal passageway). The first check valve may be configured to inhibit fluid flow in a direction from the fluid collection bag towards the catheter. The first check valve may be configured to permit fluid flow in a direction from the catheter towards the fluid collection bag. The valve body may be configured to connect the first check valve to the catheter and fluid collection bag when the valve assembly is in the first configuration. The valve body may be configured to disconnect the first check valve from the catheter and fluid collection bag when the valve assembly is in the second configuration. The valve body may be configured to connect the catheter and fluid collection bag by a tube (or a second check valve with the opposite polarity to the first check valve) when the valve assembly is in the second configuration. Thus, a simple check valve and tube may be used to operate the valve assembly. In an alternative, the first check valve could be operable to reverse its polarity in the second configuration. For example, a user operable control such as a lever or handle, could rotate the first check valve to reverse its polarity.

The valve body may be configured to move (e.g. axially) within the valve housing. The valve body may be configured to move the first check valve (e.g. axially) within the valve housing to connect and disconnect it from the proximal and distal passageways. The first check valve may be coupled to (and may move with) the button. The first check valve and tube (or second check valve) may be stacked (e.g. axially) within the valve housing. The valve body and user operable control/button may be integrally formed. As such, simple operation of the button allows the valve assembly to be operated with minimal additional components, of course, the first check valve, tube, and second check valve could be replaced with other fluidic components that operate in a similar manner to achieve the same result/benefits as the example given above.

The valve assembly may be directly connected to the fluid collection bag, for example the fluid collection bag may be directly connected to the distal passageway. Thus, the catheter assembly can form a compact unit and the operation of the valve assembly is more intuitive for the user as it is close to the bag.

The distal passageway may be connected to an access opening of the fluid collection bag. The access opening may be configured to allow fluid access to the inside of the fluid collection bag, thus the access opening may function as both an inlet and outlet of the bag. As such, any feature of an inlet or outlet described herein may equally apply to the access opening. The access opening may be formed of a flexible material. This is useful to allow the valve assembly to be moved with respect to the bag and furthermore to allow for more compact packaging of the fluid collection bag and valve assembly.

The valve assembly may be indirectly connected to the fluid collection bag, for example via an intervening tube or other fluidic component. This can allow the valve assembly to be more conveniently accessed by the user in some circumstances.

The catheter assembly may comprise a sleeve configured to enclose the catheter along the length of the catheter from the proximal end to the distal end. The sleeve may be attached to the valve assembly, preferably the sleeve is attached to the proximal passageway. The catheter and/or sleeve may be configured to deliver fluid to the valve assembly/proximal passageway. The catheter and/or sleeve may be configured to receive fluid from the valve assembly/proximal passageway. The sleeve may be configured to receive fluid from the valve assembly during draining of the fluid collection bag. The valve assembly may be directly connected to the sleeve, for example the sleeve may be directly connected to the proximal passageway. Thus, the sleeve can be used to ensure a simple and effective fluid connection between the catheter and the valve assembly.

Accordingly in one embodiment, there is provided a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag, the catheter assembly further comprising a sleeve configured to enclose the catheter along the length of the catheter from the proximal end to the distal end wherein the sleeve is configured to receive fluid from the valve assembly and deliver fluid to the valve assembly.

The fluid collection bag may comprise a panel. The fluid collection bag may comprise an inlet (for example the access opening may function as an inlet). The fluid collection bag may comprise a base. The fluid collection bag may be configured to stand up on the base. Consequently, in a broad aspect, there is provided a catheter assembly comprising a fluid collection bag and a catheter; the fluid collection bag comprising a panel, an inlet to receive fluid from the catheter, and a base, wherein the fluid collection bag is configured to stand up on the base. The fluid collection bag may comprise two panels. The fluid collection bag may comprise a peripheral bond between the two panels. The two panels may be configured to form the base. The base may be distal from the inlet.

Accordingly, a second aspect of the present invention provides a catheter assembly comprising a fluid collection bag and a catheter; the fluid collection bag comprising at least two panels, a peripheral bond between the two panels, and an inlet to receive fluid from the catheter, wherein: the two panels are configured to form a base of the fluid collection bag distal from the inlet; and the fluid collection bag is configured to stand up on the base.

Advantageously, the fluid collection bag is free-standing and is more convenient to use as it does not need to be hung or held during filling. As it is free-standing, with the inlet distal the base, the bag also fills more efficiently with liquid and can ensure that more of the bag is filled with liquid. This means the fluid collection bag can have a smaller form factor which is cheaper, lighter and more discreet for the user.

The fluid collection bag may be any suitable shape or size, for example rectangular, circular, elliptical, cuboid, spherical, etc. The fluid collection bag may comprise a front panel and a rear panel. A peripheral bond may join at least part of the periphery of the front panel and rear panel to form the fluid collection bag. The peripheral bond may define a base, two lateral edges and/or an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The fluid collection bag may have a width between the left lateral edge and right lateral edge. The width of the fluid collection bag may be at least 20, 25, 30 or 35 cm. The bag may have a width no more than 40, 35, 30, or 25 cm. Preferably, the width is between 30 and 35 cm, most preferably between 33 and 35 cm. The base may define a bottom of the bag. The upper edge may define a top of the bag. The length/height of the bag may be defined from the base to the upper edge. The height of the bag may be at least 15, 20, 25, 30, 35 or 40 cm. The height of the bag may be no more than 45, 40, 35, 30, 25, or 20 cm. The height may be between 20 and 40 cm, 25 and 40, 20 and 30 cm, 30 and 35 cm or most preferably between 33 and 35 cm, or 28 cm. Thus, preferably, the bag is square. The bag may be configured to hold at least 500, 700, or 1000 ml of fluid, and may hold no more than 1000 ml. For example, it may hold at least 700 ml of fluid.

The fluid collection bag may comprise a side panel, for example two side panels. The side panels may be configured to allow the front and rear panels to at least partially separate. Thus, the side panels allow the fluid collection bag to hold a greater volume of liquid.

Each side panel may be any suitable shape, for example lens shaped, rectangular or triangular. Preferably, the side panel is triangular, for example an isosceles triangle. Each side panel may have a height that is equal to the height of the fluid collection bag. The front panel may be joined to each side panel. The rear panel may be joined to each side panel. The front and rear panels may be joined to opposite sides of each side panel. Each side panel may have two equal length sides. The two equal length sides of a side panel may be bonded to the front and rear panels respectively. One side panel may be on a side of the fluid collection bag corresponding to the left lateral edge, and the other may be on the side corresponding to the right lateral edge. The side panels may be configured to form a base of the fluid collection bag. The side panels may be configured to allow the front and rear panels to separate further at the base of the fluid collection bag than at the upper edge of the fluid collection bag. The side panels may be configured to prevent separation of the fluid collection bag along the upper edge of the fluid collection bag. For example, the front panel and the rear panel may be directly joined to each other along the upper edge of the fluid collection bag. The two side panels may be configured to allow the front and rear panels to separate along the left lateral edge and/or right lateral edge of the fluid collection bag. The fluid collection bag may be wedge shaped. Thus, the side panels efficiently create additional volume within the bag and allow it to form a free-standing wedge shape.

The fluid collection bag may comprise a base panel. The base panel may span between the front panel and rear panel along the base of the peripheral bond. The base panel may be configured to form a base of the fluid collection bag. That is to say the base of the fluid collection bag may be formed by the front panel, the rear panel and the base panel, optionally also the two side panels. The base panel and side panels may be integrally formed. The base panel may be configured to allow the front panel and rear panel to separate to form the base of the fluid collection bag. The front panel may be joined to the base panel. The rear panel may be joined to the base panel. The front and rear panels may be joined to opposite sides of the base panel. The base may be formed by two flanges; the rear panel joined to the base panel (and/or side panels) may form one flange and the front panel joined to the base panel (and/or side panels) may form the other flange. Each flange may extend from one side of the upper edge of the peripheral bond to the other around the fluid collection bag, that is down a lateral edge (e.g. right lateral edge) along the base and back up the other lateral edge (e.g. left lateral edge). Consequently, the front panel and rear panel may be (directly) joined by the peripheral bond (only) along an upper edge of the fluid collection bag. Thus, the base panel and side panels may be used to form a base of the bag and allow it to be free-standing.

The base panel may be elongate. The base panel may comprise two elongate edges corresponding to the edges of the front and rear panels. The base panel may comprise two side edges corresponding to the edges of the two side panels. The base panel may be rectangular. The two flanges may extend from the two elongate edges. Thus, the base panel is efficiently attached to the fluid collection bag.

The base panel (and/or each side panel) may comprise a stowed configuration. In the stowed configuration, the base panel (and/or each side panel) may be configured to reduce the internal volume of the fluid collection bag. The base panel (and/or each side panel) may be folded in the stowed configuration. The base panel (and/or each side panel) may be folded between the front and rear panels. The base panel (and/or each side panel) may be configured to fold to allow the front and rear panels to come together. Thus, the base panel and/or each side panel may be folded to allow the fluid collection bag to reduce in size which assists with storage and transport of the bag making it lighter and more discreet for the user.

The base panel (and/or each side panel) may be configured to automatically unfold. The base panel (and/or each side panel) may be configured to unfold as liquid enters the fluid collection bag. Thus, the fluid collection bag can be automatically deployed into a standing configuration as liquid enters the bag, for example under the weight of liquid entering/inside the bag. This makes the bag easier to use as the user does not need to manually unfold it.

The fluid collection bag may comprise a flexible plastics material. For example, polypropylene (PP), polyethylene terephthalate (PET), low density polyethylene (LDPE), metalized polyester (MET PET) orientated polypropylene (OPP) or polyvinyl chloride (PVC). The fluid collection bag may be formed of an opaque material. At least part of the fluid collection bag may be transparent or translucent. The front panel of the fluid collection bag may be transparent or translucent. Thus, the fluid collection bag can conform to the shape of the catheter assembly, is convenient to carry and also provides a visual reference to the contents of the bag before, during and after use which makes it easier to use.

The fluid collection bag may comprise a fill level indicator. The fill level indicator may be configured to allow the user to visualise fluid within the bag. Where the fluid collection bag is formed of an opaque material, the fill level indicator may comprise a region of the fluid collection bag, such as part of the front panel, that is formed of a transparent or translucent material. The fill level indicator may be any suitable shape or size such as circular, semi-circular, triangular, rectangular or irregularly shaped, preferably it is rectangular. The fill level indicator may have a height that spans a majority of the height of the fluid collection bag, for example at least 50%, 60%, 70%, 80%, or 90% of the height of the fluid collection bag. Preferably, the fill level indicator has a height of over 90% of the height of the fluid collection bag. The fill level indicator may have a width that spans a minority of the width of the fluid collection bag, for example, no more than 50%, 40%, 30% or 20% of the width of the fluid collection bag. The fill level indicator may have a width of at least 10%, 20%, 30%, or 40% of the width of the fluid collection bag. Preferably, the fill level indicator has a width of 30-50%, or 35-40% of the width of the fluid collection bag, for example, 37%.

The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The peripheral bond may comprise any one or more of: a weld; mechanical seal; heat seal; pressure seal; adhesive; solvent bond; ultraviolet bond; ultrasonic weld; laser weld; impulse weld; or friction weld. The peripheral bond is generally permanent but in some embodiments may be re-sealable, for example through a resealable adhesive, an additional adhesive that is activated prior or during resealing, or a hook and loop/hook and hook fastener. The peripheral bond may provide a water-tight, and preferably sterile, seal. Thus the peripheral bong can be easily constructed to suit the specific needs of the catheter assembly and ensure the contents remains sterile if necessary.

The upper edge of the peripheral bond may comprise the access opening/inlet. The access opening may project out in the plane of the fluid collection bag. The access opening may project away from the fluid collection bag. The access opening may span no more than 30%, 20%, or 10% of the length of the upper edge. The access opening may be positioned at any point on the upper edge, but preferably on one side of the upper edge. The access opening may be any suitable shape, but preferably it is rectangular. Thus, the access opening is spaced from the rest of the bag which makes it easier for the user to locate and also allows it to flex and bend allowing movement of the valve assembly with respect to the fluid collection bag.

The fluid collection bag may comprise a finger hole. The finger hole may be sized to allow a user to put their finger through the finger hole and handle the bag. Thus the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. The finger hole may be any suitable shape but preferably has rounded smooth edges to allow it to be more comfortably grasped by the user. In one example, the finger hole is circular, in another it is stadium shaped, that is a rectangle with semicircles replacing two opposite sides. The finger hole may comprise two elongate straight edges with a length of one quarter to one third, for example 30%, of the height of the fluid collection bag. The elongate straight edges may be aligned parallel to the left and or right lateral edge of the fluid collection bag. The finger hole may have an aspect ratio (length:width) of at least 1:1, 2:1, 3:1, or 4:1, preferably the aspect ratio is 4:1. As an alternative to the finger hole, the fluid collection bag may comprise a series of ridges or otherwise easily gripped surface or feature in the place of the finger hole to allow the bag to be easily handled.

The finger hole may be configured to allow the bag to be rotated. Preferably, one finger hole is positioned at a corner of the bag that is opposite from the access opening/outlet. The finger hole may be adjacent to the base of the fluid collection bag. The finger hole may be closer to one lateral edge (e.g. the right lateral edge) than the access opening/outlet. The access opening/outlet may be closer to the other lateral edge (e.g. the left lateral edge) than the finger hole. Thus, the bag may be hung or held by the finger hole and gravity will drive fluid contained within the bag out through the access opening/outlet.

The fill level indicator may be positioned adjacent to the access opening/inlet/outlet, for example on the same side of the fluid collection bag as the access opening/inlet/outlet and spanning the majority of the height of the fluid collection bag. Thus, the fill level indicator ensures that during filling and draining the fluid within the fluid collection bag is visible as soon as it enters the bag and until it leaves the bag. This helps to ensure the fluid collection bag is completely emptied when draining the bag.

The fluid collection bag may comprise more than one finger hole, for example at least two, three or more finger holes. One, two, three or more, or each finger hole may have any one or more of the features of the finger hole described above. Where there is more than one finger hole, each finger hole may be located at a different corner of the bag. Thus, easy handling of the bag is facilitated through positioning of finger holes or equivalent features which also does not restrict or affect the volume of liquid that can be contained by the bag.

The access opening/outlet may be positioned distal from a finger hole of the bag. Where the access opening/outlet is positioned at or adjacent to an edge of the bag, that edge may be free from finger holes. Where the access opening/outlet is position at or adjacent an edge of the bag, a different edge of the bag (that is the right/left lateral edge) may comprise one or more finger holes. For example, the access opening/outlet may be provided at one corner of the bag and a finger hole may be provided at an opposite corner of the bag. Thus, the finger holes are positioned on edges of the bag that generally do not correspond to the position of the access opening/outlet, this makes handling/emptying of the bag easier as the bag can be rotated and held using the finger holes to allow fluid to leave the bag due to gravity.

The catheter assembly may comprise a fluid reservoir. The catheter assembly may comprise a sleeve configured to enclose the catheter along the length of the catheter from the proximal end to the distal end. The fluid reservoir may be arranged at the proximal end of the catheter. The fluid reservoir may be configured to release wetting fluid into the sleeve to wet the catheter. The sleeve may be in fluid communication with the fluid collection bag.

Accordingly a third aspect of the present invention there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag. Consequently, excess wetting fluid may flow from the sleeve into the fluid collection bag.

Advantageously, the present invention provides for a catheter assembly which benefits from the introduction of wetting fluid at the proximal end of the catheter which reduces the risk of discomfort and injury while using the catheter as the proximal end, which enters the body first, is adequately wetted prior to use. In addition, as the wetting fluid is contained within the sleeve and flows from the proximal end to the distal end, the fluid is used efficiently to wet the whole length of the catheter by prioritising the parts of the catheter that enter the body first. Due to the sleeve being in fluid communication with the fluid collection bag, wetting fluid cannot escape outside the sleeve and as such the user's hands or clothing does not inadvertently contact the wetting fluid which could make handling the catheter difficult. This ensures that the catheter is easy and convenient to use, as well as being discrete and hygienic to dispose of as the wetting fluid is contained within the sleeve/bag.

The sleeve may comprise a flexible plastics material. The sleeve may be liquid impermeable. The sleeve may comprise a thermoplastic polyurethane (TPU) or low-density polyethylene (LDPE). Thus, the sleeve is cheap and easy to produce and can be easily manipulated by the user during use.

The fluid reservoir may be configured to retain a wetting fluid within it. The fluid reservoir may be configured to release wetting fluid to activate the catheter. The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter. The fluid reservoir may be configured to retain at least 5, 10, 15, 25, or 50 ml of wetting fluid.

The fluid reservoir may comprise a deformable, frangible or burstable sachet. A deformable, frangible or burstable reservoir enhances the convenience for a user as fluid can be easily released without additional tools. A sachet in particular can be more easily handled on an assembly line compared to handling fluid itself. The fluid reservoir may comprise a flexible plastics material, for example, a thermoplastic polyurethane (TPU) or low-density polyethylene (LDPE).

The fluid reservoir may be configured to retain at least 5, 10, 15, 25, or 50 ml of wetting fluid. The fluid reservoir may be configured to retain the wetting fluid within it. The fluid reservoir may be configured to retain at least 5, 10, 15, 25, or 50 ml of wetting fluid, most preferably at least 12 ml of fluid. The fluid reservoir may be configured to release wetting fluid into the sleeve; in particular the fluid reservoir may be configured to release wetting fluid between an internal surface of the sleeve and an external surface of the catheter. The fluid reservoir may be configured to retain and/or release excess wetting fluid. Excess wetting fluid is defined herein as more than is normally required to activate/wet the catheter, for example 10%, 20%, 50% or more, in excess of that which would normally be provided to wet the catheter (having regard for example to the length of the catheter and the composition of the wetting fluid). The sleeve may be configured to allow excess wetting fluid to flow into the fluid collection bag. Thus, the reservoir may release an excess of wetting fluid to ensure that the catheter is well wetted and excess fluid can flow into the fluid collection bag ensuring it does not negatively affect subsequent use of the catheter.

The catheter and/or sleeve may be in fluid communication with the fluid collection bag via the valve assembly and preferably connected to the proximal passageway. The proximal passageway may be configured to allow fluid to enter the valve assembly from the distal end of the catheter and optionally via the sleeve. The proximal passageway may be connected to one end of the sleeve which corresponds to the distal end of the catheter. Thus, the proximal passageway allows liquid to pass into the fluid collection bag.

The catheter assembly may be configured to allow fluid to pass directly from the sleeve to the proximal passageway and/or directly from the catheter to the proximal passageway. One lateral side (e.g. a right lateral side) of the catheter may be attached to one side (e.g. a right side) of the proximal passageway. There may be a gap between a different lateral side (e.g. a left lateral side) of the catheter and a respective different side (e.g. a left side) of the proximal passageway. The distal end of the catheter may be within the proximal passageway. The sleeve may be sealed around a periphery of the proximal passageway. As such, fluid travelling within the sleeve is directed into the proximal passageway directly via the gap. In addition, fluid travelling within the catheter passes out the distal end and into the proximal passageway. In other examples, the catheter may be positioned differently and may be attached, for example, to the left side of the proximal passageway.

The catheter assembly may be configured to allow fluid to pass from the catheter into the sleeve. One lateral side (e.g. a right lateral side) of the catheter may be attached to a side (e.g. right side) of the sleeve. A distal end of the catheter may be within the sleeve. A distal end of the catheter may not be within the proximal passageway. The sleeve may be sealed around a periphery of the proximal passageway. As such, fluid travelling within the sleeve is directed into the proximal passageway directly and fluid travelling within the catheter passes out the distal end into the sleeve and then from there into the proximal passageway. In other examples, the catheter may be positioned differently and may be attached to a left side of the sleeve (or indeed a front side or rear side). Furthermore, the distal end of the catheter may be blocked and an outlet may be provided in a lateral side of the catheter to allow fluid to pass out into the sleeve.

The catheter assembly may be configured to allow fluid to pass from the sleeve into the catheter. One lateral side (e.g. a right lateral side) of the catheter may be attached to one side (e.g. a right side) of the proximal passageway. The proximal passageway may be sealed around the catheter on all sides. Consequently, there may be no gap between the proximal passageway and the catheter. As such, fluid may only pass from the sleeve/catheter into the proximal passageway if it is within the catheter. A side, e.g. a lateral side of the catheter may comprise a sleeve intake opening. The sleeve intake opening may be any suitable shape such as oval or circular. The sleeve intake opening may be adjacent to the proximal passageway. The sleeve intake opening may permit the passage of fluid from the sleeve into the catheter. Thus fluid may enter the catheter from the sleeve to join fluid travelling within the catheter. From here, the fluid may pass out the distal end of the catheter and into the proximal passageway. Many other configurations may be possible to achieve the same effect, for example, the sleeve may be attached to the outer sides of the catheter itself.

The sleeve may comprise a reservoir adapter. An end of the sleeve may comprise a reservoir adapter. The reservoir adapter may be configured to engage the fluid reservoir. The reservoir adapter may be configured to receive wetting fluid from the fluid reservoir. The reservoir adapter may be configured to detach from the fluid reservoir. The reservoir adapter may be configured to permit the catheter to pass through the reservoir adapter.

According to a broad aspect of the present invention, there is provided catheter assembly comprising a fluid reservoir, a catheter which comprises a proximal end for insertion into the body and a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The catheter assembly may be a packaged catheter assembly comprising a pouch containing the fluid reservoir and catheter.

According to a fourth aspect of the present invention, there is provided a packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

Thus, wetting fluid is efficiently and easily introduced to wet the catheter as the reservoir adapter retains the fluid reservoir at one end of the sleeve. This ensures that wetting fluid is not wasted and also that wetting fluid is less likely to leak out and wet the external surface of the sleeve or pouch which may cause discomfort for the user.

The sleeve may comprise a proximal end and a distal end corresponding to the proximal end and distal end of the catheter respectively. The proximal end of the sleeve may comprise the reservoir adapter. The reservoir adapter may be positioned at or adjacent to the proximal end of the catheter. Thus, the reservoir is adapted to deliver wetting fluid to the proximal end of the catheter first, this ensures that the part of the catheter first introduced into the body is most likely to be adequately wetted. This reduces the risk of injury or discomfort during use of the catheter.

The reservoir adapter may annular, for example it may be an O-ring. The reservoir adapter may comprise a bore therethrough. The bore may be sized to allow the catheter to pass through the reservoir adapter. The bore may be sized to allow the catheter and wetting fluid to pass through the reservoir adapter. The reservoir adapter may be configured to inhibit passage of the catheter through the reservoir adapter/bore while the fluid reservoir is engaged by the reservoir adapter. The fluid reservoir may be configured to block the catheter from passing through the reservoir adapter/bore. The fluid reservoir may be configured to block the catheter from passing through the reservoir adapter/bore if the fluid reservoir is engaged by the reservoir adapter. Thus, the catheter may be withdrawn from the sleeve through the reservoir adapter and bore, but only once the fluid reservoir is removed from the reservoir adapter. This helps to protect the catheter from dirt and damage prior to use.

The reservoir adapter may be configured to engage the fluid reservoir by any suitable means, such as push-fit, bayonet fit, or screw-fit engagement. The reservoir adapter may have an internal size and shape that matches the fluid reservoir. Thus, the fluid reservoir may be easily push-fit into the reservoir adapter.

The fluid reservoir may comprise a housing. The housing may be configured to surround the sachet, preferably the housing is tight fitting on all sides of the sachet. The housing may be configured to direct wetting fluid released from the sachet to wet the catheter. The housing may be fluid impermeable. The housing may be flexible. The housing may be formed from any suitable material such as silicone or a flexible plastics material like thermoplastic polyurethane (TPU) or low-density polyethylene (LDPE). The housing may be substantially the same shape as the sachet, for example rectangular. The housing may have rounded edges, this can help to ensure fluid does not get trapped in corners of the housing.

The reservoir adapter may engage the housing. The reservoir adapter may engage the housing via a wetting tube positioned at the proximal end of the sleeve. The housing may be integrally formed with the wetting tube. The housing may be a separate object from the wetting tube. The housing may be engaged by the wetting tube in a variety of ways such as push-fit, screw-fit or through an adhesive or weld, preferably the housing is push-fit into the wetting tube. The sleeve may extend around the wetting tube. The reservoir adapter may be fitted around the sleeve. The reservoir adapter may be fitted around the wetting tube. The reservoir adapter may be fitted around the housing. The reservoir adapter may urge any two or more of the sleeve, wetting tube and housing into engagement with one another. The Thus, the reservoir adapter can provide a simple but effective fluid tight seal between the fluid reservoir and sleeve.

The reservoir adapter may have an inner diameter that is less than an outer diameter of the wetting tube. The reservoir adapter may have an inner diameter that is less than an outer diameter of the housing. The reservoir adapter may be formed of a material that is more flexible than the wetting tube. The sleeve may be formed of a material that is more flexible than the reservoir adapter and/or wetting tube. Thus, the reservoir adapter stretches to accommodate the wetting tube and does not significantly reduce the size of the wetting tube when it is engaged by the reservoir adapter. This ensures fluid flow from the fluid reservoir to the sleeve is not unnecessarily inhibited.

The fluid reservoir may be configured to release wetting fluid when activated by a user. The fluid reservoir may comprise an activation marker. The fluid reservoir may be configured to release wetting fluid upon activation of the activation marker. The activation marker may be on a surface of the fluid reservoir, such as on the housing. The activation marker may comprise a region of the fluid reservoir that is identifiable to the user, for example through visual and/or tactile feedback. Preferably, the activation marker may be at least visually recognisable, for example a different colour, shade of colour, or pattern, to the rest of the fluid reservoir. Thus, the activation marker is easily recognisable while the fluid reservoir is engaged by the reservoir adapter.

The pouch may comprise two walls (a front wall and a rear wall). The pouch may comprise a peripheral seal between the two walls. When the pouch is sealed it may be in a closed configuration. The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. As outlined above, the peripheral seal may provide a sterile seal. Both the fluid collection bag and peripheral seal of the pouch may provide a sterile seal. Thus the peripheral seal can be easily constructed to suit the specific needs of the pouch and ensure the contents remains sterile within the pouch. Advantageously, additional protection is provided to ensure the catheter remains sterile allowing the pouch to be opened to provide a sterile surface which, if used and made dirty, does not affect the sterility of the catheter.

The peripheral seal may form one or more edges of the pouch. A folded edge may comprise the fold between the two walls (the first wall and the second wall). All edges other than the folded edge may be formed by the peripheral seal. Preferably, the peripheral seal comprises all edges of the pouch. The peripheral seal may comprise a base, a left lateral edge, right lateral edge and an upper edge. The left and right lateral edges may be defined as the left and right edges when viewing the pouch in the closed configuration with the front wall in front of the rear wall, the base at the bottom and the upper edge at the top.

The pouch may have a width defined as the distance between the left lateral edge and right lateral edge of the pouch of at least 60, 70, 80, 90, 100, 120, 140, 160 or 180 mm. The pouch may have a width of no more than 200, 180, 160, 140, 120, 100, 90, 80, or 70 mm. Preferably, the width is between 60-110 mm, and most preferably about 90-110 mm, for example 105 mm. The pouch may have a height defined as the distance between the upper edge and base (i.e. the height when in the closed configuration) of at least 60, 70, 80, 90, 100, 120, 140, 160, 180, 200, 220, 240 or 250 mm. The pouch may have a height of no more than 250, 240, 220, 200, 180, 160, 140, 120, 110, 100, 90, 80 or 70 mm. Preferably, the height is between 100-250 mm, or 120-170 mm and most preferably it is about 1500 mm. Thus, the pouch is a compact shape when closed so that it is convenient and discreet for the user to carry, for example it is easier to fit in a bag or pocket.

The folded edge may be configured to resist being broken. A frangible portion of the peripheral seal may form one or more edges of the pouch that do not comprise the folded edge, in particular, all the edges other than the folded edge may form the frangible portion. The frangible portion may be configured to break before the folded edge. The pouch may comprise only one folded edge.

The pouch may contain any one or more of the fluid collection bag and catheter. The fluid collection bag may be provided in a stowed configuration while inside the pouch. Thus, the size of the fluid collection bag is minimised inside the pouch and this assists the user in removing the bag and other elements of the packaged catheter assembly from the pouch.

The pouch may comprise a flexible plastics material. For example, polypropylene (PP), polyethylene terephthalate (PET), low density polyethylene (LDPE), metalized polyester (MET PET) or orientated polypropylene (OPP). The pouch may be formed from an opaque material. The pouch may have a printed appearance or may be matte. This reduces the impression that it is a medical device and thus make it more discreet to carry.

The pouch may comprise an interaction region defining a first point of contact for a user of the packaged catheter assembly. The interaction region may facilitate user access to the contents of the pouch. Actuation of the interaction region may create an opening in the pouch. The interaction region may be a break-away region such as a peel-away or tear-away region configured to be at least partially separated from the pouch. The catheter may be withdrawn through the opening. The catheter may be withdrawn through the opening. Before withdrawal of the catheter from the pouch, the opening may comprise a sterile seal. The sterile seal may be broken by actuation of the interaction region, for example by peeling a peel-away region from the pouch.

The break-away region may form a part of the walls of the pouch, for example, the break-away region may form a part of the front wall and/or rear wall. Preferably, the break-away region is positioned adjacent to a lateral edge (e.g. right lateral edge) of the pouch. The break-away region may be any suitable shape or size, for example, rectangular, circular, ellipsoidal or irregularly shaped. Preferably, the break-away region is rectangular. The break away region may be defined by a tear line along which the pouch preferentially tears. The tear line may comprise a line of weakness in the walls of the pouch. The tear line may be formed by any suitable means, for example laser scoring. The tear line may span between two edges of the pouch, for example the upper edge and base. The tear line may be present on the front and/or rear wall of the pouch, preferably just the front wall. A tear start may be provided at a first end of the tear line. The tear start may comprise a notch, or area of weakness, in one or both of the walls of the pouch. The tear start may define the first part of the pouch to be torn. A tear stop may be provided at a second end of the tear line. The tear stop may act to prevent or reduce the likelihood of the tear-away region being completely separated from the remainder of the packaging. Thus, the break-away region can be used to form an opening in the pouch in a controlled manner. In addition, the tear line can only span one wall of the pouch which prevents the break-away region from becoming fully separated from the pouch which may be a nuisance for the user as they would then need to retain and dispose of an additional loose item.

The tear line may be flexibly positioned on the pouch. The tear line may span between two points on the edge of the pouch. The tear start may be positioned on an edge of the pouch. The tear stop may be positioned on an edge of the pouch. The tear start and tear stop may be positioned on opposite edges of the pouch, preferably the upper edge and base respectively. The tear line may be positioned off-center with respect to the width of the pouch. Preferably, the tear line is positioned 70-90% across the width of the pouch from one lateral edge to the other, (e.g. left lateral edge to right lateral edge). The break-away region may therefore correspond to 30-10% of the width of the pouch. As such, a break-away region is formed which is a minority of the width of the pouch and this ensures that the contents of the pouch do not fall out in an uncontrolled manner as soon as the pouch is opened.

The break-away region may be the same colour, a different shade of colour or a different colour to the rest of the pouch. Thus, the break-away region can either be made more discreet or more obvious depending on the needs of the packaged catheter assembly.

The break-away region may comprise a pull-ring. The pull ring may be arranged adjacent to the tear start. The pull ring may be sized to allow a finger to pass through it. The pull ring may be configured to tear the pouch along the tear line. Thus, the pull ring makes it easier for the user to open the pouch by providing a ring which they can easily grip and pull, of course a ring itself may not be necessary and instead any feature the user can easily grasp could be used.

The pull ring may be a different colour, or shade of colour, from the walls of the pouch. Thus, it is more easily identifiable for the use.

The break-away region may comprise an opening marker configured to provide guidance to the user about how to open the pouch. The opening marker may direct the user to tear the pouch along the tear line, for example by grasping and pulling on the pull ring. The opening marker may comprise an arrow. The arrow may point from the tear start towards the tear stop along the tear line. The opening marker may provide visual guidance to the user, or may provide tactile guidance or otherwise for example using a region of the pouch that is a different texture to the rest of the pouch.

The fluid collection bag may be contained within the pouch. The fluid collection bag may be provided in a stowed configuration, preferably while inside the pouch. The fluid collection bag may be configured to remain in the stowed configuration. In the stowed configuration, the fluid collection bag may be folded or rolled to reduce its size in any one dimension. The stowed configuration may be a folded or rolled configuration. Preferably, the fluid reservoir is folded along its width and height when in the stowed configuration. Thus, the fluid collection bag is stored in a compact manner that allows the pouch to also be a small size and convenient for the user to carry on their person.

The access opening may be provided in a folded configuration within the pouch. The valve assembly may overlap the fluid collection bag within the pouch. The fluid reservoir may be contained within the pouch. The fluid reservoir may be positioned adjacent to the valve assembly in the pouch, for example beneath the valve assembly. The catheter may be provided in a curved and/or coiled configuration. The distal end of the catheter may be arranged adjacent to the valve assembly/proximal passageway. The proximal end of the catheter may be arranged adjacent to the fluid reservoir/reservoir adapter/wetting tube. The sleeve may comprise a turn between the wetting tube and the proximal end of the catheter. The turn may be at least 90 degrees, 120 degrees, 150 degrees or 180 degrees, preferably the turn is 180 degrees. Preferably, the turn is a hairpin turn. Thus, the packaged catheter assembly is arranged in a compact manner that is easy to access and use.

The fluid reservoir may be in a position within the pouch corresponding to the tear line. The fluid collection bag may be in a position within the pouch corresponding to the tear line. Thus, when the tear line is torn, the fluid reservoir and fluid collection bag are accessible for the user to grasp and remove from the pouch.

The catheter assemblies/packaged catheter assemblies of the first to fourth aspects may include any one or more features of a catheter assembly/packaged catheter assembly as defined in general/broad terms, or according to any other of the first to fourth aspects set out above. The catheter assemblies/packaged catheter assemblies of the first to fourth aspects may comprise any of the optional features of the others of the first to fourth aspects without necessarily including all the features required of them. That is to say, an optional feature which happens to be set out following one particular aspect does not necessarily apply only to that aspect, so, for example, the disclosure provides for a packaged catheter assembly comprising a pouch containing the catheter assembly of the first aspect, that is, a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag; wherein the fluid collection bag comprises two side panels as described in relation to the second aspect and wherein the pouch comprises a break-away region comprising a pull ring as described in relation to the third aspect.

According to a fifth aspect of the present invention there is provided a method of manufacturing a catheter assembly comprising the steps of providing a catheter having a proximal end for insertion into the body and a distal end and a fluid collection bag, providing a valve assembly and arranging it between the catheter and fluid collection bag such that the fluid collection bag may receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

The method of the fifth aspect of the invention may be a method of manufacturing the catheter assembly of the first aspect of the invention, which, of course, may include any optional feature outlined above.

The method may comprise providing the valve assembly in the first configuration. Thus, the valve assembly may allow the user to fill the fluid collection bag and use the catheter without first needing to change the configuration of the valve assembly.

According to a sixth aspect of the present invention there is provided a method of manufacturing a catheter assembly comprising the steps of providing a catheter having a proximal end for insertion into the body and a distal end and forming a fluid collection bag, the fluid collection bag being formed by providing two panels, creating a peripheral bond between the two panels, and providing an inlet in the fluid collection bag to receive fluid from the catheter, wherein: the two panels are configured to form a base of the fluid collection bag distal from the inlet; and the fluid collection bag is configured to stand up on the base.

The method of the sixth aspect of the invention may be a method of manufacturing the catheter assembly of the second aspect of the invention, which, of course, may include any optional feature outlined above.

The method may comprise providing a base panel. The method may comprise joining the base panel to the front and/or rear panels of the fluid collection bag. The method may comprise folding the base panel. The base panel may be folded along a line parallel to a base edge of the front and/or rear panels.

The method may comprise providing two side panels. The method may comprise joining each side panel to the front and/or rear panels of the fluid collection bag. The method may comprise joining each side panel to the base panel. The method may comprise folding each side panel. Thus, the interior volume of the fluid collection bag may be further increased.

According to a seventh aspect of the present invention there is provided a method of forming a catheter assembly comprising the steps of providing a catheter having a proximal end for insertion into the body and a distal end, arranging a fluid collection bag to receive fluid from the distal end of the catheter, enclosing the catheter along the length of the catheter from the proximal end to the distal end with a sleeve, and arranging a fluid reservoir at the proximal end of the catheter, wherein the fluid reservoir is configured to release wetting fluid into the sleeve to wet the catheter and wherein the sleeve is in fluid communication with the fluid collection bag.

The method of the seventh aspect of the invention may be a method of forming the catheter assembly of the third aspect of the invention, which, of course, may include any optional feature outlined above.

According to an eighth aspect of the present invention there is provided a method of forming a packaged catheter assembly comprising the steps of providing a fluid reservoir, a catheter, and a pouch, and arranging the fluid reservoir and catheter within the pouch; wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The method of the eighth aspect of the invention may be a method of forming the packaged catheter assembly of the fourth aspect of the invention, which, of course, may include any optional feature outlined above.

The method may comprise arranging the reservoir adapter at the end of the sleeve. The method may comprise inserting the fluid reservoir into the reservoir adapter so as to block/inhibit the passage of the catheter therethrough. Thus, the catheter is protected by the fluid reservoir/reservoir adapter.

The method may comprise forming a housing around a sachet. The method may comprise attaching the housing to the end of the sleeve using the reservoir adapter.

According to a ninth aspect of the present invention there is provided a method of draining a fluid collection bag of a catheter assembly, the catheter assembly comprising a catheter having a proximal end for insertion into the body and a distal end, a valve assembly, and a fluid collection bag arranged to receive fluid from the distal end of the catheter via the valve assembly, the method comprising switching the valve assembly from a first configuration in which the valve assembly inhibits fluid flow in a direction from the fluid collection bag towards the catheter and permits fluid flow in a direction from the catheter towards the fluid collection bag to a second configuration in which the valve assembly permits fluid flow in a direction from the fluid collection bag towards the catheter and inhibits fluid flow in a direction from the catheter towards the fluid collection bag, and allowing the fluid collection bag to drain through the valve assembly.

The method of ninth aspect of the present invention may be a method of preparing the catheter assembly of the first aspect of the invention, which, of course, may include any optional feature outlined above and may be manufactured according to the fifth aspect of the invention.

The method may comprise draining the fluid collection bag through the catheter. The method may comprise draining the fluid collection bag through a sleeve configured to enclose the catheter. Thus, the sleeve and catheter may be used to direct the flow of liquid out of the fluid collection bag.

The method may comprise introducing the catheter by its proximal end into the urethra before draining the fluid collection bag. The method may comprise allowing fluid to pass from the body and into the fluid collection bag via the catheter before draining the fluid collection bag. Consequently, the method may be a method of using a catheter assembly.

According to a tenth aspect of the present invention there is provided a method of preparing a catheter assembly for use, the catheter assembly comprising a catheter and the fluid collection bag, the fluid collection bag comprising two panels, a peripheral bond between the two panels, and an inlet to receive fluid from the catheter, the method comprising using the two panels to form a base of the fluid collection bag distal from the inlet; and standing the fluid collection bag up on the base.

The method of tenth aspect of the present invention may be a method of preparing the catheter assembly of the second aspect of the invention, which, of course, may include any optional feature outlined above and may be manufactured according to the sixth aspect of the invention.

The method may comprise separating the two panels (i.e. the front and rear panels) to form the base. The method may comprise unfolding a base panel of the fluid collection bag. The method may comprise introducing liquid into the fluid collection bag. The method may comprise introducing liquid into the fluid collection bag to automatically form the base in the fluid collection bag.

The method may comprise introducing the catheter by its proximal end into the urethra. The method may comprise allowing fluid to pass from the body and into the fluid collection bag via the catheter. Consequently, the method may be a method of using a catheter assembly.

According to an eleventh aspect of the present invention there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve.

The method of the eleventh aspect of the invention may be a method of wetting the catheter of the catheter assembly of the third aspect of the invention, which, of course, may include any optional feature outlined above and may be manufactured according to the seventh aspect of the invention.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to a twelfth aspect of the present invention, there is provided a method of unpacking a packaged catheter assembly, the packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to engage the fluid reservoir, the method comprising the steps of releasing wetting fluid from the fluid reservoir into the sleeve, and detaching the fluid reservoir to permit the catheter to pass through the reservoir adapter.

The method of the twelfth aspect of the invention may be a method of wetting a catheter from the packaged catheter assembly of the fourth aspect of the invention, which, of course, may include any optional feature outlined above and may be manufactured according to the eighth aspect of the invention.

The method may comprise forming an opening in the pouch. The step of releasing wetting fluid may comprise rupturing the fluid reservoir, for example by pressing the wetting marker. The method may comprise allowing fluid to flow through the reservoir adapter. Thus, the fluid reservoir may be easily activated to release wetting fluid into the sleeve.

The fluid reservoir may be detached from the reservoir adapter after the release of wetting fluid from the fluid reservoir. Thus, the catheter is adequately wetted before the fluid reservoir is removed.

The method may comprise withdrawing the catheter from the sleeve. Only part of the catheter, preferably including the proximal end of the catheter, may be withdrawn from the sleeve. The method may comprise passing the catheter through the reservoir adapter. The method may comprise bunching the sleeve. The catheter may be withdrawn from the sleeve after it has been wetted. The catheter may be withdrawn from the sleeve after the fluid reservoir has been removed from the sleeve. Thus, the fluid reservoir protects the catheter before use, and the catheter is easily withdrawn through the reservoir adapter.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

The methods of the fifth to twelfth aspects of the present invention may of course individually include any one or more of the features, optional or otherwise, of one another and further may include any one or more of the features, optional or otherwise, of the first to fourth aspects of the present invention.

In any aspect, a catheter assembly is preferably a closed catheter assembly, that is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments of the invention may also be open catheter assemblies in that fluid is not collected or retained by the catheter assembly.

### DETAILED DESCRIPTION OF THE INVENTION

In order that the invention may be more clearly understood one or more embodiments thereof will now be described, by way of example only, with reference to the accompanying drawings, of which:

FIG. 1 is a front view of a packaged catheter assembly where the pouch is in the closed configuration;

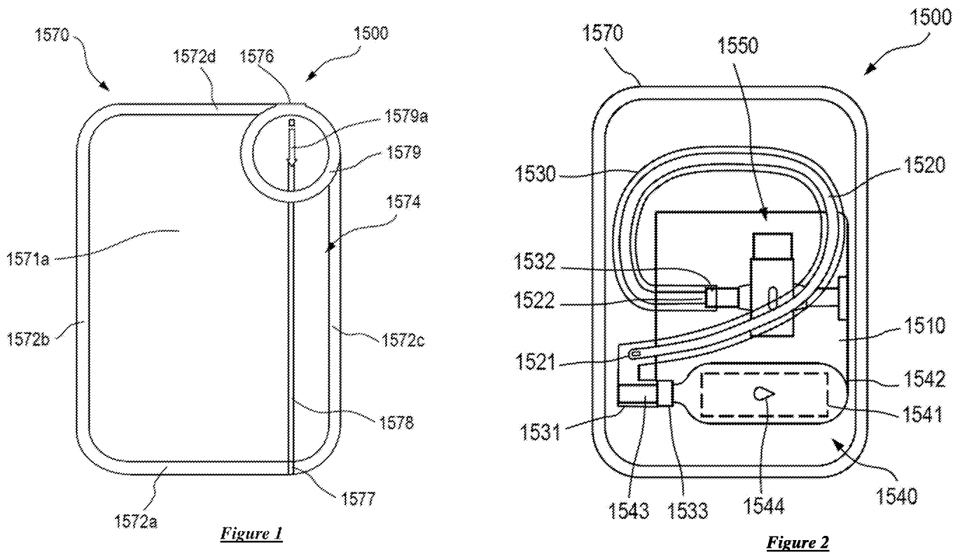

FIG. 2 is a front cut-away view of the packaged catheter assembly of FIG. 1;

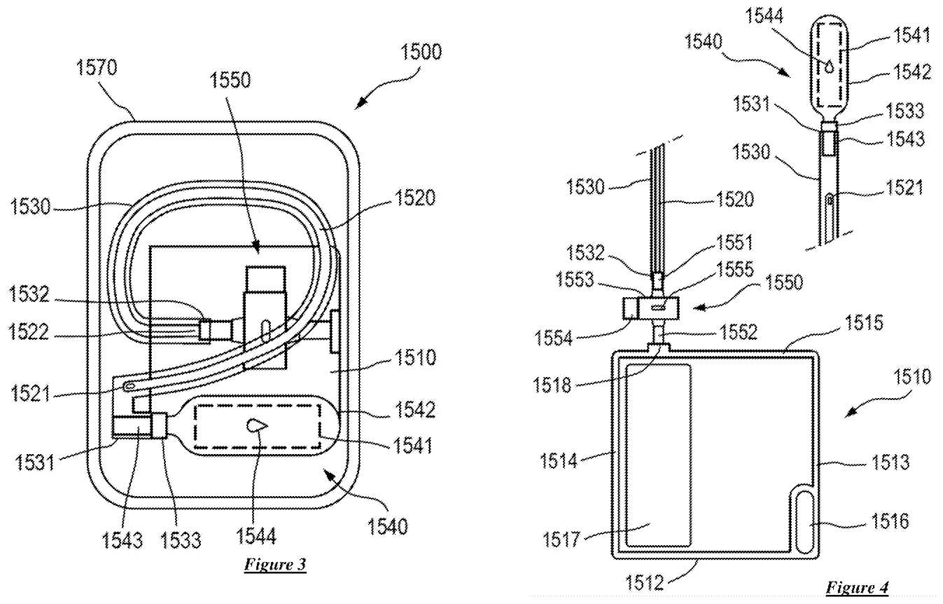

FIG. 3 is a front view of the packaged catheter assembly of FIG. 1 where the pouch is in the open configuration and the catheter assembly contained within the pouch is being removed from the pouch;

FIG. 4 is a front view of the catheter assembly from the packaged catheter assembly of FIG. 1;

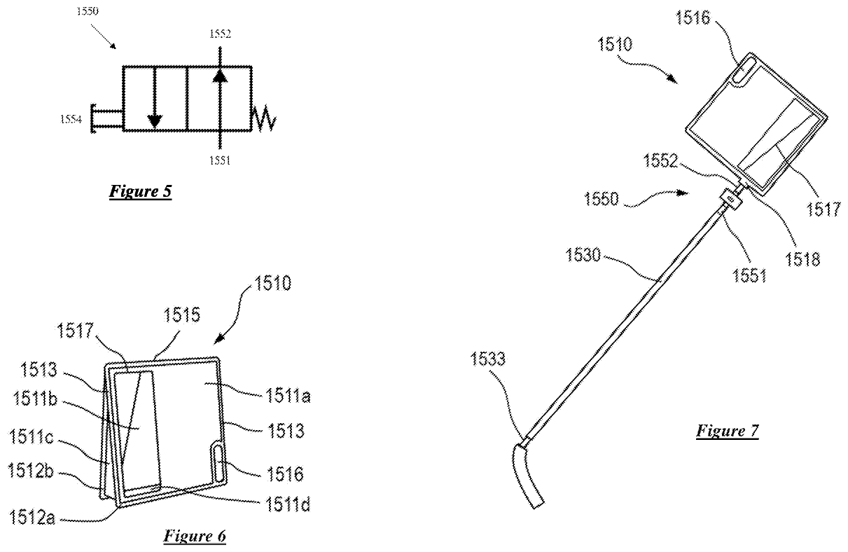

FIG. 5 is a schematic diagram of showing the operation of the valve assembly of the packaged catheter assembly of FIG. 1;

FIG. 6 is a perspective view of the fluid collection bag of the packaged catheter assembly of FIG. 1;

FIG. 7 is a front view of liquid being drained from the fluid collection bag of the packaged catheter assembly of FIG. 1;

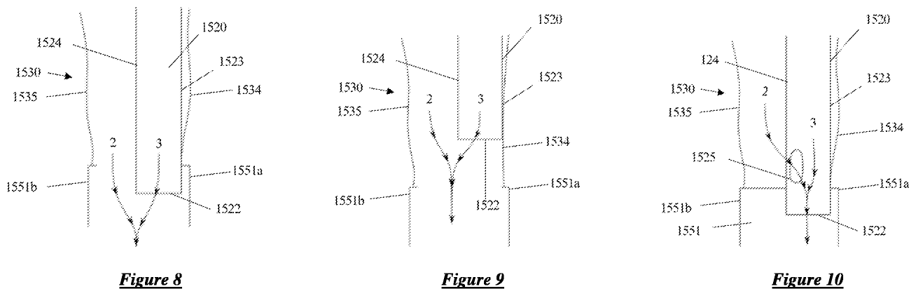

FIG. 8 is a cut-away view of a first example of the inlet of the catheter assembly of FIG. 1;

FIG. 9 is a cut-away view of a second example of the inlet of the catheter assembly of FIG. 1;

FIG. 10 is a cut-away view of a third example of the inlet of the catheter assembly of FIG. 1;

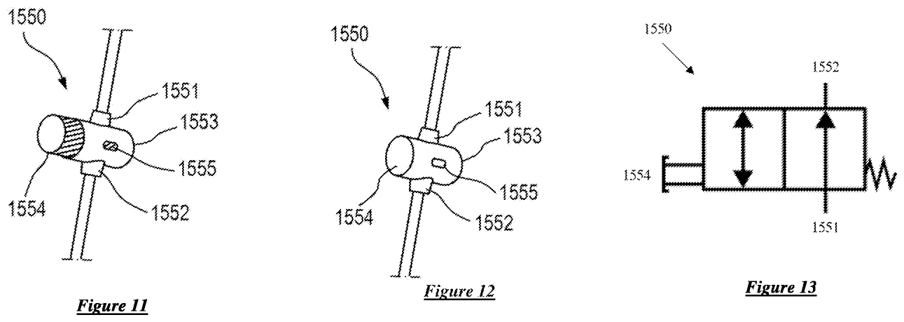

FIG. 11 is a perspective view of a variation on the valve assembly of the packaged catheter assembly of FIG. 1 in the first configuration;

FIG. 12 is a perspective view of the valve assembly of FIG. 11 in the second configuration; and

FIG. 13 is a schematic diagram of showing the operation of the valve assembly of FIG. 11.

Referring to FIGS. 1-10, a first embodiment of a packaged catheter assembly 1500 is shown. In the Figures, as is conventional, dashed lines indicate hidden features.

The packaged catheter assembly 1500 comprises a catheter assembly contained within a pouch 1570. The catheter assembly comprises a fluid collection bag 1510, a catheter 1520, a sleeve 1530 and a fluid reservoir 1540. The catheter 1520 has a proximal end 1521 for insertion into the body and a distal end 1522, and the fluid collection bag 1510 is arranged to receive fluid from the distal end 1522 of the catheter 1520, as such the catheter assembly is a “closed catheter assembly”.

In this embodiment, the catheter 1520 is a male urinary catheter made from a hydrophilic thermoplastic elastomer (TPE). The sleeve 1530 of this embodiment is a thermoplastic polyurethane (TPU) or low-density polyethylene (LDPE). Obviously those skilled in the art will be able to select suitable alternative examples.

The pouch 1570 is formed from a front wall 1571a and rear wall 1571b of identical shape and size, and a peripheral seal joining the periphery of the walls to form the pouch 1571 and containing its contents (i.e. the catheter assembly).

The peripheral seal defines a base 1572a, a left lateral edge 1572b, a right lateral edge 1572c and an upper edge 1572d of the pouch 1570. The left lateral edge 1572b and right lateral edge 1572c being defined as the left and right sides of the pouch 1500 when viewing the pouch 1570 with the rear wall 1571b behind the front wall 1571a, the base 1572a at the bottom of the pouch 1570 and the upper edge 1572d at the top of the pouch 1570. The peripheral seal thus defines a pouch 1570 that is generally rectangular with rounded corners and suitably has a width between the lateral edges of between 60-110 mm, preferably 90-110 mm, for example about 105 mm, and a height from the base 1572a to the upper edge 1572d of between 100 to 250 mm, preferably 120 to 170 mm, for example about 150 mm. The base 1572a defines the bottom of the pouch 1570 in use, and the upper edge 1572d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 1570 may be formed from a single piece of material. The pouch may then be formed by folding this single piece of material along a pouch fold line to form the walls. In addition, in some embodiments any number of folds may be used to form the pouch 1570. For example, none of the edges may comprise a fold (as in the illustrated example), one edge may comprise a fold, or two or more of the edges may comprise a fold.

In this embodiment, the pouch 1570 is formed from an opaque material and as such provides a discrete package for its contents which can make the user more comfortable in carrying the packaged catheter assembly 1500 in day-to-day life. In addition, the exterior of the pouch 1570 may have a textured or printed appearance, for example a matte appearance, and feel to reduce the impression that it is a medical device.

In this embodiment, the pouch 1570 also comprises an interaction region which is arranged adjacent to the right lateral edge 1572c of the pouch 1570 on the front wall 1571a. The interaction region is used to provide access to the pouch through/near to the right lateral edge 1572c. In this embodiment, the interaction region comprises a rectangular break-away region 1574. The break-away region 1574 comprises a tear start 1576 at the point the upper edge 1572d meets the break-away region 1575, a tear stop 1577 at a corresponding point on the base 1572a, and a tear line 1578 spanning between the tear start 1576 and tear stop 1577. The tear line 1578 comprises a line of weakness in the walls of the pouch 1570 configured to preferentially tear and allow the break-away region to be at least partially separated from the pouch 1570. The tear line 1578 may be formed by any suitable means but in this embodiment is laser etched.

In this embodiment, the tear line 1578 is positioned off-center with respect to the width of the pouch 1570. The tear line 1578 is positioned 70-90% across the width of the pouch 1570 from the left lateral edge 1572b to the right lateral edge 1572c, for example 80% across. In other embodiments, the tear-away region may have a different shape or size as required by the shape/size of the pouch 1570 or the contents of the pouch 1570. For example, it may be positioned centrally with respect to the width and/or may be arranged in a different orientation such that access is provided through a corner of the pouch 1570 or a different edge of the pouch 1570.

In this embodiment, the tear line 1578 extends across the front wall 1571a only and not the rear wall 1571b. As such, once the tear line 1578 is torn, the break-away region 1574 remains attached to the pouch 1570 by the rear wall 1571b. This makes the pouch 1570 more convenient to use as the break-away region doesn't become a separate item and so the user doesn't need to handle/store/dispose of an extra item once the tear line 1578 is activated. In other embodiments, the break-away region 1574 may be configured to completely separate from the pouch 1570. This could be advantageous to allow easier access to the pouch 1570.

In this embodiment, the break-away region 1574 comprises a pull-ring 1579 arranged adjacent to the tear start 1576. The pull ring 1579 is sized to allow a finger to pass through and grip then pull so as to tear the front wall 1571a along the tear line 1578 and create an opening in the pouch 1570. Of course, in other embodiments the pull ring 1579 may be replaced by a different kind of easily gripped object, such as a tab.

In this embodiment, the pull-ring 1579 is a different colour from the front wall 1571 of the pouch 1570 to allow the user to more easily recognise it and open the pouch 1570. In addition, an opening marker 1579a in the form of an arrow is present on the front wall 1517a in a position corresponding to the pull-ring 1579 and tear line 1578 and pointing the towards the base 1572a of the pouch 1570. The opening marker 1579a is the same colour as the pull-ring 1579. The user is therefore guided to open the pouch 1570. Of course, in other embodiments, the pull-ring may be a different shade of colour from the pouch, or may be the same colour/shade of colour where discretion is prioritised. In addition, the opening marker may not be present or may have a different form. In some embodiments, it may comprise a region which is a different texture to the pouch for example where the user is visually impaired.

In this embodiment, the fluid reservoir 1540 is configured to retain a wetting fluid and release wetting fluid to activate the surface of the catheter 1520. In this embodiment, the wetting fluid is water and interacts with the hydrophilic surface of the catheter 1520 to render it lubricious. In other embodiments, other wetting fluids may be used and they may be polar (e.g. water-based) or non-polar (e.g. oil-based) depending on the catheter's surface properties. In this embodiment, the fluid reservoir 1540 is configured to retain and release 12 ml of wetting fluid, of course in other embodiments more or less wetting fluid may be required.

In this embodiment, the fluid reservoir 1540 comprises a burstable rectangular sachet 1541 configured to retain 5 to 50 ml of wetting fluid, for example 12 ml. In this embodiment, the fluid reservoir also comprises a housing 1542 that surrounds the sachet 1541. The housing 1542 comprises a flexible and fluid impermeable material such as soft silicone. In other embodiments, the housing 1542 may comprise a thermoplastic polyurethane (TPU) or low-density polyethylene (LDPE). The housing 1542 is configured to enclose the sachet 1415 on all sides and is tight fitting on all sides, as such the housing 1542 is also generally rectangular in shape but with rounded edges.

In this embodiment, the housing 1542 is push-fit into a wetting tube 1543 at one end that extends away from the fluid reservoir 1540, of course in other embodiments screw fittings or seals such as a weld may join the housing 1542 to the wetting tube 1543. In this embodiment, the housing 1542 has an aspect ratio (length:width:height) of 5:2:2 and as such is elongate. The wetting tube 1543 connects to a middle of a face of the housing 1542 that is perpendicular to the length of the housing 1542. In this embodiment, the wetting tube 1543 extends in a direction parallel to the length of the housing 1542 and is approximately one third the length of the housing 1542 with a circular cross-section and a diameter approximately half the width and/or height of the housing 1542. The face of the housing 1542 on the opposite side from the wetting tube 1543 is rounded to the extent that it forms a hemispherical end of the housing 1542.

The user can release wetting fluid held by the fluid reservoir 1540 by compressing the fluid reservoir 1540, for example by pinching the housing 1452 between their fingers. To facilitate the user in activating the fluid reservoir 1540, the housing 1542 comprises a wetting marker 1544. The wetting marker 1544 comprises a region of the housing 1542 that is a different colour or texture to the rest of the housing 1542 to allow the user to easily identify the position of the sachet 1451 and where to press to activate it and release wetting fluid. In other embodiments, the fluid reservoir 1540 may be configured to release wetting fluid through a different means, for example by twisting or bursting the fluid reservoir in a different way.

The sleeve 1530 of this embodiment is sized to enclose the catheter 1520 along the majority of the length of the catheter 1520, and preferably the entire length of the catheter 1520. The sleeve 1530 comprises a proximal end 1531 corresponding to the proximal end 1521 of the catheter 1520 and a distal end 1532 corresponding to the distal end 1522 of the catheter 1520. The distal end 1532 of the sleeve 1530 is attached to a valve assembly 1550 as described below. The proximal end 1531 of the sleeve is configured to receive and engage the wetting tube 1543 such that wetting fluid released by the fluid reservoir 1540 is directed into the sleeve 1530 as described further below.

In this embodiment, the proximal end 1531 of the sleeve 1530 comprises a reservoir adapter 1533 configured to retain the fluid reservoir 1540 at the proximal end 1531 of the sleeve 1530. In this embodiment, the reservoir adapter 1533 achieves this by retaining the wetting tube 1543 and housing 1542 in engagement with the sleeve 1530. The reservoir adapter 1533 is fitted around the sleeve 1530 and wetting tube 1543 and comprises an O-ring with an inner diameter that is slightly less than the outer diameter of the wetting tube 1543. Consequently, when the reservoir adapter 1533 is fitted around the wetting tube 1543 and proximal end 1531 of the sleeve 1530, the reservoir adapter 1533 urges the sleeve 1530 against the wetting tube 1543. Thus, a fluid-tight seal is formed between the sleeve 1530 and wetting tube 1543 and the fluid reservoir 1540 is retained in position via the housing 1542.

To ensure tight engagement between the wetting tube 1543 and sleeve 1530, the sleeve 1530 should be formed from a material that is more flexible than both the wetting tube 1543 and reservoir adapter 1533. Preferably, the reservoir adapter 1533 is more flexible than the wetting tube 1543 to ensure the wetting tube 1543 is not compressed when engaged by the reservoir adapter 1533. If the wetting tube 1543 is compressed, this could lead to an unwanted reduction or restriction of wetting fluid flow from the fluid reservoir 1540 to the sleeve 1530. In this embodiment, the reservoir adapter 1533 comprises rubber, however, in other embodiments, a different material may be used such as silicone.

In this embodiment, the reservoir adapter 1533 is configured to release the fluid reservoir 1540 and housing 1542 to allow the proximal end 1521 of the catheter 1520 to pass out of the sleeve 1530 and be used. In this embodiment, the housing 1542 can be released from the reservoir adapter 1533 by being simply pulled out of the sleeve 1530. In other embodiments, specific fittings or tear-lines may be used to allow the housing 1542 to be released from the reservoir adapter 1533. In this embodiment, the wetting tube 1543 is not part of the fluid reservoir 1540 and thus, when the housing 1542 is removed from the sleeve 1530, the wetting tube 1543 remains in the proximal end of the sleeve 1530 to be used as a handling element. In other embodiments, the wetting tube 1543 may be integrally formed with the housing 1542 and may be removed from the sleeve 1530 with the housing 1542.

In this embodiment, the valve assembly 1550 is connected to the distal end 1532 of the sleeve 1530 and the fluid collection bag 1510 and is configured to control the flow of fluids between them. In this embodiment, the valve assembly 1550 comprises two configurations: a first configuration in which the valve assembly 1550 inhibits fluid flow in a direction from the fluid collection bag 1510 towards the catheter 1520 and permits fluid flow in a direction from the catheter 1520 towards the fluid collection bag 1510; and a second configuration in which the valve assembly 1550 permits fluid flow in a direction from the fluid collection bag 1510 towards the catheter 1520 and inhibits fluid flow in a direction from the catheter 1520 towards the fluid collection bag 1510. Thus, the valve assembly 1550 allows the user to both fill and empty the fluid collection bag 1510 via the sleeve 1530 in a controlled manner as described below.

In this embodiment, the valve assembly 1550 comprises a proximal passageway 1551 configured to provide a fluid communication between the valve assembly 1550 and the distal end 1532 of the sleeve 1530 and the catheter 1420, and a distal passageway 1552 configured to provide a fluid connection between the valve assembly 1550 and the fluid collection bag 1510. The connection between the sleeve 1530 and/or catheter 1520 and the proximal passageway 1551 is flexible as described further below. Each passageway 1551, 1552 connects to opposite sides of a side wall of a cylindrical valve housing 1553 and the valve assembly 1550 is configured to control the fluid flow between the passageways 1551, 1552 as shown in FIG. 5 and described below. The valve housing 1553 has a diameter of approximately 1 to 3 cm, for example 2 cm and a length approximately double its diameter. In this embodiment, each passageway 1551, 1552 extends away from the valve housing 1553 by a distance that is not more than the diameter of the valve housing 1553. Each passageway 1551, 1552 is tubular with an internal diameter that is approximately equal to the internal diameter of the catheter 1520, thus each passageway 1551, 1552 has a similar fluid resistance to the catheter 1520 and does not significantly inhibit the flow of fluid into/out of the fluid collection bag 1510.

In this embodiment, one end of the valve housing 1553 is open and is filled by a user operable control which comprises a button 1554 of cylindrical shape. The button 1554 is configured to allow the user to select the first or second configurations of the valve assembly 1550 and may be depressed in an axial direction of the valve housing 1553. In one example, the button 1554 may be in an extended configuration when the valve assembly 1550 is in the first configuration and a depressed configuration when the valve assembly 1550 is in the second configuration. In the extended configuration, the button 1554 extends beyond the open end of the valve housing 1553 and in the depressed configuration, the button 1554 is flush with the open end of the valve housing 1553. The user can switch between the configurations by depressing the button 1554 past the depressed configuration, for example an audible/tactile click may signal that the button 1554 has been sufficiently depressed, for example via a protrusion (not shown) on the inside of the valve housing 1553 that overrides a detent (not shown) on the button 1554 as the button 1554 moves with respect to the valve housing 1553. The user operable control also comprises a biasing element in the form of a spring (not shown) to bias the user operable control and ensure that the button 154 is always returned to the correct extended configuration. Thus, the valve assembly 1550 may be operated using a simple button and can be readily operated by someone with reduced dexterity by simply squeezing the button in their hand. Of course, other configurations of the button 1554 may be readily implemented by the skilled person or indeed a different type of user operable control may be used such as a switch, button, lever, slider, knob. Thus, the valve can be flexibly operated as required by the user.

In this embodiment, the valve housing 1554 comprises a window 1555 located in the side wall of the valve housing 1554 between the passageways 1551, 1552. The window 1555 may be configured to indicate to the user if the valve assembly 1550 is in the first or second configuration as described further below. The window 1555 is elongate with a length parallel to the axis of the valve housing 1554. The window 1555 has a length of approximately half the diameter of the valve housing 1554 and the window has an aspect ratio (length:width) of approximately 3:1. The window is stadium-shaped that is rectangular semicircles replacing two opposite sides.

In this embodiment, the side wall of the button 1554 is a different colour from the valve housing 1553 and a top end of the button 1554 (that is the end that is outside the valve housing 1553) comprises an activation marker 1556. In this embodiment, the activation marker 1556 comprises the words “PUSH” to provide visual guidance on how to operate the valve assembly 1550. In other embodiments, the side wall of the button may be a different shade of colour from the valve housing, and/or the entire button may be a different colour/shade of colour from the valve housing. This can help the user to identify the button 1554 and use the valve assembly 1550.

In addition, the button 1554 is visible through the window 1555 described above. In this embodiment, the parts of the button 1554 that are visible through the window 1555 when the button 1554 is in the extended and depressed configurations respectively are different colours or different shades of colour. This helps the user to identify which configuration the valve assembly 1550 is in. In this embodiment, the window 1555 appears to be the same colour as the button 1554 when the button 1554 is in the extended configuration and is a different colour from the button 1554 when the button 1554 is in the depressed configuration. The user is thereby given a visual indication of the state of the valve assembly 1550.

Inside the valve housing 1553 and between the passageways 1551, 1552, there is a valve body (not shown) that is configured enable the valve assembly 1550 to operate as shown in FIG. 5. The valve body is configured to inhibit fluid flow in a direction from the fluid collection bag 1510 and distal passageway 1552 towards the catheter 1520 and proximal passageway 1551 and permit fluid flow in a direction from the catheter 1520 and proximal passageway 1551 towards the fluid collection bag 1510 and distal passageway 1552 when the button 1554 is in the extended configuration. The valve body is also configured to permit fluid flow in a direction from the fluid collection bag 1510 towards the catheter 1520 and inhibits fluid flow in a direction from the catheter 1520 towards the fluid collection bag 1510 and distal passageway 1552 when the button 1554 is in the depressed configuration. Thus, the valve body enables the valve assembly 1550 to function as required.

The valve body could be any suitable arrangement of fluidic components as would be familiar to the skilled person to enable the valve assembly 1550 to operate as described above. In one example, the valve body comprises two opposing check valves in series: a first check valve to prevent fluid flow in a direction from the catheter 1520 and proximal passageway 1551 towards the fluid collection bag 1510 and distal passageway 1552, and a second check valve to prevent fluid flow in a direction from the fluid collection bag 1510 and distal passageway 1552 towards the catheter 1520/proximal passageway 1551, for example spring loaded in-line check valves could be used for the first and second check valves. The button 1554 is then configured to inhibit the operation of the second check valve in the extended configuration and the first check valve when in the depressed configuration, for example by physically preventing the respective check valve from closing. In a different example, the valve body may comprise two opposing check valves as described above but stacked on top of one another in parallel within the valve housing and coupled to the button 1554. Movement of the button 1554 between the extended and depressed configurations may then select which check valve is in fluid communication with the passageways 1551, 1552 and thus alter the polarity of the valve assembly 1550. Thus, the button 1554 and valve body provide the first and second configurations of the valve assembly 1550. Of course, in other embodiments, a lever, switch or other user operable input may be used to control the valve assembly 1550 and a different type of valve body may be used to achieve the same effect.

In this embodiment, the distal passageway 1552 is connected directly to an access opening 1518 of the fluid collection bag 1510, that is without any intervening components such as tubing. The access opening 1518 is configured to allow fluid to flow into and out of the fluid collection bag 1510 as required. In other embodiments, the distal passageway may be connected indirectly to the access opening of the fluid collection bag 1510a, for example via an intervening section of tubing or other fluid connection device placed between the distal passageway and fluid collection bag.

In this embodiment, the fluid collection bag 1510 comprises a front panel 1511a, a rear panel 1511b of identical shape and size, and a peripheral bond joining the periphery of the panels to form the bag 1510. The peripheral bond defines a base 1512, a right lateral edge 1513, a left lateral edge 1514 and an upper edge 1515. The right lateral edge 1513 and left lateral edge 1514 being defined as the right and left sides of the bag 1510 when viewing the bag 1510 with the rear panel behind the front panel 1511, the base 1512 at the bottom of the bag 1510 and the upper edge 1515 at the top of the bag 1510. The peripheral bond thus defines a bag 1510 that is generally rectangular and preferably square with a width between the lateral edges of between 25 and 40 cm, 30 and 35 cm, or most preferably 33 and 35 cm, and a height from the base 1512 to the upper edge 1515 of between 25 and 40 cm, 30 and 35 cm, or most preferably 33 and 35 cm. The bag 1510 is thus configured to hold at least 700 ml of fluid released from the user through the catheter safely. The base 1512 defines the bottom of the bag 1510 in use, and the upper edge 1515 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the fluid collection bag comprises two side panels 1511c and a base panel 1511d. The side panels 1511c are each triangular shaped in the form of elongate isosceles triangles with a height equal to the height of the fluid collection bag 1510. The two equal length sides of each side panel 1511c are bonded to the front and rear panels 1511a, 1511b respectively with one side panel 1511c separating the front and rear panels 1511a, 1511b along the left lateral edge 1514 of the fluid collection bag 1510 and the other separating the front and rear panels 1511a, 1511b along the right lateral edge 1513.

The base panel 1511d that spans between the front panel 1511a and rear panel 1511b along the base 1512 of the peripheral bond and between the base edges of the side panels 1511c. The base panel 1511d is therefore rectangular shaped with a width equal to the width of the fluid collection bag 1510. The base panel 1511d has a length that is equal to the length of the base edge of the side panels 1511c and the base panel 1511d has an aspect ratio (length:width) of approximately 1:4. The base panel 1511d and side panels 1511c thus allow the front panel 1511a and rear panel 1511b to separate along the base 1512 and form a wedge-shaped fluid collection bag 1510.

Consequently, the base panel 1511d and side panels 1511c allow the fluid collection bag 1510 to have a greater internal volume and also to be free-standing when partially filled with fluid as described below.

The base 1512 is formed by two flanges 1512a, 1512b; the rear panel joined to the base panel by the peripheral bond forms a first flange 1512a and the front panel joined to the base panel by the peripheral bond forms the second flange 1512b. The two flanges 1512a, 1512a extend around the left lateral edge 1514, base 1512 and right lateral edge 1513 of the fluid collection bag 1510 where the side panels 1511c and base panel 1511d meets the rear panel 1511b and the front panel 1511a respectively.

In this embodiment, the panels comprise a flexible plastics material, for example polypropylene (PP), polyethylene terephthalate (PET), low density polyethylene (LDPE), metalized polyester (MET PET) orientated polypropylene (OPP), polyvinylpyrrolidone (PVP), or polyvinyl chloride (PVC). Consequently, the panels 1511a, 1511b, 1511c, 1511d may be folded or rolled to reduce the physical size of the fluid collection bag 1510 before, during or after use. In this embodiment, the fluid collection bag 1510 is provided in a stowed configuration before use, which is a folded configuration as described in more detail below.

In this embodiment, the corner of the peripheral bond where the right lateral edge 1514 and base 1512 meet comprises a finger hole 1516 to allow the bag 1510 to be easily handled by a user. The finger hole 1516 is stadium shaped, that is a rectangle with semicircles replacing two opposite sides. The finger hole 1516 comprises two elongate straight edges with a length of one quarter to one third, for example 30%, of the height of the fluid collection bag 1510 and aligned parallel to the right lateral edge 1514. The finger hole 1516 has an aspect ratio (length:width) of approximately 4:1. In other embodiments, the finger hole may comprise a series of ridges, or otherwise easily gripped surface or feature to allow the user to easily handle the bag. In addition, there may be different numbers of finger holes, such as two or more, and they may be placed in different locations on the bag.

In this embodiment, the panels 1511a, 1511b, 1511c, 1511d are formed from an opaque material. This makes the fluid collection bag 1510 more discreet for the user. Of course, in other embodiments, any one or more of the panels may be transparent or translucent.

In this embodiment, the fluid collection bag 1510 further comprises a fill level indicator 1517. The fill level indicator 1517 comprises a region of the front panel 1511a that is formed from a transparent or translucent material configured to allow a user to visualise the amount of liquid within the fluid collection bag 1510. The fill level indicator spans a majority of the height of the fluid collection bag 1510 and in this embodiment spans over 90% of the height of the fluid collection bag 1510. In this embodiment, the fill level indicator 1517 has a width of approximately 30-50% the width of the fluid collection bag 1510, for example 35-40% or most preferably 37%. Thus, the fill level indicator 1517 is a rectangular window that can be used to visualise the contents of the fluid collection bag 1510.

In this embodiment, the fill level indicator 1517 does not contain any fill level markers to allow the fill level of the fluid collection bag 1510 to be quantitatively measured. Of course, in other embodiments, fill level markers may be present for example to let the user identify when the fluid collection bag 1510 is a certain % full, for example the fill level markers may indicate 25%, 50%, and 75% fill levels of the fluid collection bag.

Referring to FIG. 7, in this embodiment, the finger hole 1516, fill level indicator 1517 and access opening 1518 are positioned with respect to one another to enhance the usability of the fluid collection bag 1510.

In this embodiment, the access opening 1518 is positioned in the upper edge 1515 of the fluid collection bag 1510 at a point approximately 15-30% along the upper edge 1515 from the left lateral edge 1514 to the right lateral edge 1513. Thus, the access opening 1518 is positioned at or near an opposite corner of the fluid collection bag 1510 from the finger hole 1516. This allows the user to easily grasp the finger hole 1516 and allow the fluid collection bag 1510 to drain completely through the access opening 1518 while the bag 1510 hangs from the finger hole 1516 and without further manipulation.

In this embodiment, the fill level indicator 1517 spans the majority of the height of the fluid collection bag 1517 and is positioned adjacent to the left lateral edge 1514 of the fluid collection bag 1517. Thus, the fill level indicator 1517 is on the same side of the fluid collection bag 1517 as the access opening 1518 and ensures that during filling and draining the fluid within the fluid collection bag 1510 is visible as soon as it enters the bag and until it leaves the bag. This helps to ensure the fluid collection bag 1510 is completely emptied when draining the bag.

Referring to FIGS. 2 and 3, in this embodiment, the fluid collection bag 1510 is provided in a stowed folded configuration as mentioned above. The fluid collection bag 1510 is folded once at a mid-point along its width and then again at a midpoint along its height thus in the stowed configuration the fluid collection bag 1510 is a square with a width/height half its unfolded width/height. When contained within the pouch 1570, the access opening 1518 is also folded such that the valve assembly 1550 is positioned to overlie the fluid collection bag 1510 and minimise the physical size of the catheter assembly. The valve assembly 1550 is initially provided in the first configuration and as such permits flow of fluids from the catheter 1520 into the fluid collection bag 1510, in addition, the button 1554 is in the extended configuration.

The position and configuration of the catheter 1520 is not particularly limited so long as it is not curved to the extent that damage to the catheter is likely, for example from kinking. In this embodiment, the catheter 1520 is arranged in a curved/coiled configuration, this helps ensure that the catheter 1520 is not excessively curved and minimises risk of damage.

In this embodiment, the fluid reservoir 1540/housing 1542 is positioned adjacent to the base 1572a of the pouch 1570 and beneath the valve assembly 1550. The wetting tube 1543 is therefore positioned adjacent to the left lateral edge 1572b of the pouch 1570. The sleeve 1530 extends from the wetting tube 1543 and completes a hairpin turn before enclosing the proximal end 1521 of the catheter 1520. This takes advantage of the flexible nature of the sleeve 1530 which can be arranged with tight turns/folds to reduce the size of the catheter assembly. This is because even if the sleeve 1530 becomes kinked, kinks in the sleeve 1530 are unlikely to negatively affect the usability of the catheter assembly. It is of course important that any such tight turns/folds are present in a portion of the sleeve 1530 that does not enclose the catheter 1520 as kinks in the catheter 1520 may render it unusable.

Prior to use, the pouch 1570 is provided sealed closed with the contents arranged within it as mentioned above. The contents of the pouch 1570 can be accessed by opening the pouch 1570. The pouch 1570 is opened by grasping the pouch 1570 in one hand and pulling the pull-ring 1579 in the direction indicated by the opening marker 1579a. This breaks the front wall 1571a of the pouch 1570 along the tear line 1578 from the tear start 1576 in the upper edge 1572d to the tear stop in the base 1572a. Thus, an opening is created and the break-away region 1574 is partially removed from the pouch 1570 allowing the catheter assembly to be removed from the pouch 1570 as shown in FIG. 3.

Once the pouch 1570 has been opened, the fluid collection bag 1510, catheter 1520 and sleeve 1530 may be removed from the pouch 1570. To ensure the catheter 1520 is wetted prior to use, the user must activate the fluid reservoir 1540 by squeezing the housing 1542 at the position indicated by the wetting marker 1544. Preferably, the user visualises the wetting fluid leaving the housing 1542 through the wetting tube 1543 and into the sleeve 1530. The wetting fluid enters the sleeve 1530 and flows down the sleeve 1530 wetting the catheter 1520 from its proximal end 1521 to its distal end 1522. This prioritises wetting of the proximal end 1521 and ensures that it is wetted prior to use which is important to reduce the likelihood of injury during use as the proximal end 1521 is the first part of the catheter 1520 to be introduced into the body.

The wetting fluid may then flow through the valve assembly 1550 and into the fluid collection bag 1510 where it is collected.

Before use, the fluid collection bag 1510 may be unfolded to ensure that the collection of liquid within it is not hindered by folds in the fluid collection bag 1510. In addition, the fluid collection bag 1510 may be opened such that the side panels 1551c and base panel 1511d are unfolded and the fluid collection bag 1510 is free-standing on its base. This can be helpful as the user does not need to hold or support the fluid collection bag 1510 and as the access opening 1518 is on the upper edge 1515 of the fluid collection bag 1510, fluid will efficiently fill the bag while it remains standing upright.

The fluid reservoir 1540 and housing 1542 initially block the catheter 1520 from leaving the sleeve 1530 and as such, the user must then remove the housing 1542 from the sleeve by grasping the reservoir adapter 1533 in one hand and pulling the housing 1542 away from it with the other. The reservoir adapter 1533 then separates from the wetting tube 1543 leaving the wetting tube 1543 in place within the sleeve 1530 and restricted by the reservoir adapter 1533. Once the housing 1542 has been removed, the proximal end 1521 of the catheter 1520 may emerge from the sleeve 1530 and be progressively introduced into the body until urine flows therethrough and into the fluid collection bag 1510 via the valve assembly 1550. In this embodiment, the relatively rigid wetting tube 1543 provides a handling element that can be used to guide the proximal end 1521 of the catheter 1520 into the body without having to touch the catheter 1520 directly and risk infection.

Referring to FIG. 7, after use of the catheter 1520, the user may wish to empty the contents of the fluid collection bag 1510. This can be desirable in order to reduce the size and weight of the used bag 1510 and/or to dispose of the bag 1510. To allow the bag 1510 to be emptied, the valve assembly 1550 must be switched into the second configuration by depression of the button 1554 to the depressed configuration. The fluid collection bag 1510 may also be grasped and lifted by the finger hole 1516 to allow gravity to drive fluid flow out of the access opening 1518, through the valve assembly 1550 and out through the catheter 1520/sleeve 1530. The wetting tube 1543 may once again be used to guide the flow of fluid out of the sleeve 1530 to minimise the risk of spillages.

Advantageously, the valve assembly 1550 may be used to selectively start and stop fluid flow through switching between the first and second configurations using the button 1554. This can allow the user to better control draining to minimise leaks/spillage of fluids, for example, they may hang the fluid collection bag 1510 in an elevated position and arrange the sleeve 1530/catheter 1520 into a receptacle such as a drain or toilet before depressing the button 1554 and allowing fluid to drain out of the fluid collection bag 1510.

As mentioned above, in this embodiment the catheter 1520 and sleeve 1530 are in fluid communication with the proximal passageway 1551. The configuration of the catheter 1520, sleeve 5130, and proximal passageway 1551 is relatively flexible and many different configurations could be employed in embodiments of the invention.

Referring to FIG. 8, in a first example of the configuration for the catheter 1520, sleeve 1530 and proximal passageway 1551 fluid passes directly from the sleeve 1530 to the proximal passageway 1551 and directly from the catheter 1520 to the proximal passageway 1551. In this example, a right lateral side 1523 of the catheter 1520 is attached to a right side 1551a of the proximal passageway 1551 and there is a gap between a left lateral side 1524 of the catheter 1520 and the respective left side 1551b of the proximal passageway 1551. In addition, the distal end 1522 of the catheter 1520 is within the proximal passageway 1551, and the sleeve 1530 is sealed around a periphery of the proximal passageway 1551. As such, fluid travelling within the sleeve 2 is directed into the proximal passageway 1551 directly via the gap between the left lateral side 1524 of the catheter 1520 and the left side 1551b of the proximal passageway 1551. In addition, fluid travelling within the catheter 3 passes out the distal end 1522 and into the proximal passageway 1551 directly. In other examples, the catheter 1520 may be positioned differently and may be attached to the left side 1551b of the proximal passageway 1551, or to a front or rear side.

Referring to FIG. 9, in a second example fluid passes from the catheter 1520 into the sleeve 1530 and then into the proximal passageway 1551. In this example, the right lateral side 1523 of the catheter 1520 is attached to a left side 1534 of the sleeve 1530 and a distal end 1522 of the catheter 1520 is not within the proximal passageway 1551. As in the first example, the sleeve 1530 is sealed around a periphery of the proximal passageway 1551. As such, fluid travelling within the sleeve 2 is directed into the proximal passageway 1551 directly and fluid travelling within the catheter 3 passes out the distal end 1522 into the sleeve 1530 and then from there into the proximal passageway 1551. In other examples, the catheter 1520 may be positioned differently and may be attached to a left side 1535 of the sleeve 1530, or indeed to a front or rear side. Furthermore, the distal end 1522 of the catheter 1520 may be blocked and an outlet (not shown) may be provided in a lateral side of the catheter 1520 to allow fluid to pass out into the sleeve 1530.

Referring to FIG. 10, in a third example fluid passes from the sleeve 1530 into the catheter 1520 and then into the proximal passageway 1551. The configuration of this example is similar to the first example shown in FIG. 8 and so only the differences are described. In this example, the proximal passageway 1551 is sealed between the left side 1551b and right side 1551a such that the catheter 1520 is sealed on all sides. As such, fluid may only pass from the sleeve 1530/catheter 1520 into the proximal passageway 1551 if it is within the catheter 1520. The left lateral side 1524 of the catheter 1520 comprises an oval sleeve intake opening 1525 adjacent to the proximal passageway 1551 which permits the passage of fluid travelling within the sleeve 3 into the catheter to join fluid travelling within the catheter 2. From here, the fluid 2, 3 passes out the distal end 1522 of the catheter 1520 and into the proximal passageway 1551. Many other configurations may be possible to achieve the same effect, for example, the sleeve 1530 may be attached to the outer sides of the catheter 1520 itself.

Referring to FIGS. 11 to 13, a variation on the valve assembly 1550 described above is shown. In this example, the valve assembly 1550 is positioned in-line between two sections of tubing rather than directly connected to a fluid collection bag as with the first embodiment. This could be advantageous to enable more compact or flexible packaging of the catheter assembly, or to position the valve assembly 1550 in a more convenient place for the user to interact with.

In this example, the valve assembly 1550 is configured such that in the second configuration fluid flow is permitted in both directions, that is from the catheter 1520 towards the fluid collection bag 1510 and vice versa. This retains the core function of the valve assembly 1550 to selectively allow the fluid collection bag 1510 to be drained by switching from the first to second configuration, but does so with a slightly simplified design as one-way flow is not required in the second configuration.

In this example, the valve body could therefore be a single check valve arranged to prevent fluid flow from the fluid collection bag towards the catheter. The button 1554 may be configured to inhibit the check valve from closing when the valve assembly 1550 is in the second configuration thus enabling two-way flow.

The one or more embodiments are described above by way of example only. Many variations are possible without departing from the scope of protection afforded by the appended claims.

1. A catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

2. The catheter assembly of claim 1 wherein the catheter is a male urinary catheter.

3. The catheter assembly of any preceding claim wherein the valve assembly is configured to move from the first configuration to the second configuration and/or from the second configuration to the first configuration.

4. The catheter assembly of claim 3 wherein the valve assembly comprises a user operable control configured to move the valve assembly from the first from the first configuration to the second configuration and/or from the second configuration to the first configuration and the user operable control comprises a button.

5. The catheter assembly of claim 4 wherein the valve assembly comprises a valve housing and the button comprises a side wall that is a different colour or shade of colour from the valve housing.

6. The catheter assembly of claim 6 wherein the side wall is visible when the side wall of the button is in the first configuration and the side wall of the button is hidden by the valve housing when the valve assembly is in the second configuration.

7. The catheter assembly of any preceding claim wherein the valve assembly comprises a valve housing and the valve housing comprises a window, wherein the window may be configured to change appearance depending on if the valve assembly is in the first configuration or second configuration.

8. The catheter assembly of any proceeding claim wherein the catheter is configured to receive fluid from the valve assembly and deliver fluid to the valve assembly.

9. The catheter assembly of any proceeding claim further comprising a sleeve configured to enclose the catheter along the length of the catheter from the proximal end to the distal end wherein the sleeve is configured to receive fluid from the valve assembly and deliver fluid to the valve assembly.

10. The catheter assembly of any preceding claim wherein the valve assembly is directly connected to the fluid collection bag.

11. The catheter assembly of any preceding claim wherein the valve assembly comprises a proximal passageway configured to provide a fluid communication between the valve assembly and the catheter and a distal passageway configured to provide a fluid connection between the valve assembly and the fluid collection bag, and a valve housing.

12. The catheter assembly of claim 11 wherein the valve housing comprises a valve body fluidly connected to the proximal and distal passageways.

13. The catheter assembly of claim 12 wherein the valve body is coupled to a user operable control comprising a button and the valve body is configured to move axially within the valve housing.

14. A packaged catheter assembly comprising a pouch and the catheter assembly of any preceding claim, wherein the pouch is configured to contain the catheter assembly within it.

15. A method of manufacturing a catheter assembly comprising the steps of providing a catheter having a proximal end for insertion into the body and a distal end and a fluid collection bag, providing a valve assembly and arranging it between the catheter and fluid collection bag such that the fluid collection bag may receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

16. The method of claim 15 wherein the valve assembly is provided in the first configuration.

17. A method of draining a fluid collection bag of a catheter assembly, the catheter assembly comprising a catheter having a proximal end for insertion into the body and a distal end, a valve assembly, and a fluid collection bag arranged to receive fluid from the distal end of the catheter via the valve assembly, the method comprising switching the valve assembly from a first configuration in which the valve assembly inhibits fluid flow in a direction from the fluid collection bag towards the catheter and permits fluid flow in a direction from the catheter towards the fluid collection bag to a second configuration in which the valve assembly permits fluid flow in a direction from the fluid collection bag towards the catheter and inhibits fluid flow in a direction from the catheter towards the fluid collection bag, and allowing the fluid collection bag to drain through the valve assembly.

18. The method of claim 17 further comprising draining the fluid collection bag through a sleeve configured to enclose the catheter.

19. The method of claim 17 or 18 further comprising draining the fluid collection bag through the catheter.

20. The method of any of claims 15 to 19 wherein the catheter assembly is a catheter assembly according to any of claims 1 to 14.
